(19) United States
(12) Patent Application Publication
(10) Pub. No.: US 2011/0275998 A1
(43) Pub. Date: Nov. 10, 2011

(54) SYSTEMS AND METHODS FOR DELIVERING A THERAPEUTIC AGENT

(75) Inventors: J. Richard Gyory, Sudbury, MA (US); Daniel Hamilton, Mont Vernon, NH (US); Alessandro Pizzochero, Chelmsford, MA (US); Jon Taylor, Groton, MA (US)

(73) Assignee: SPRINGLEAF THERAPEUTICS, INC., Boston, MA (US)

(21) Appl. No.: 13/102,695

(22) Filed: May 6, 2011

Related U.S. Application Data
(60) Provisional application No. 61/332,066, filed on May 6, 2010.

(51) Int. Cl. A61M 37/00

(52) U.S. Cl. 604/134; 604/131

(57) ABSTRACT

Devices and methods for delivering a fluid to a patient are disclosed herein. In one embodiment, a delivery system includes a reservoir for containing a fluid and a fluid communicator in fluid communication with the reservoir. An electrochemical actuator is coupled to the reservoir and configured to exert a force on the reservoir upon actuation such that fluid within the reservoir is communicated through the fluid communicator. The actuator includes a first end that is constrained and a second end that is not constrained. The actuator is configured to bend at a location along a length of the actuator when actuated such that the second end of the actuator is displaced in a direction toward the fluid reservoir. The actuator can be an electrochemical actuator. The apparatus can further include a transfer structure disposed between the actuator and the reservoir configured to contact the reservoir upon actuation of the actuator.

### CROSS-REFERENCE TO RELATED APPLICATIONS

This application claims priority to and the benefit of U.S. Provisional Application Ser. No. 61/332,066, filed May 6, 2010, entitled “Systems And Methods For Delivering a Therapeutic Agent,” the disclosure of which is hereby incorporated by reference herein in its entirety.

### BACKGROUND

The invention relates generally to medical devices and procedures, including, for example, medical devices and methods for delivering a therapeutic agent to a patient.

Drug delivery involves delivering a drug or other therapeutic compound into the body. Typically, the drug is delivered via a technology that is carefully selected based on a number of factors. These factors can include, but are not limited to, the characteristics of the drug, such as drug dose, pharmacokinetics, complexity, cost, and absorption, the characteristics of the desired drug delivery profile (such as uniform, non-uniform, or patient-controlled), the characteristics of the administration mode (such as the ease, cost, complexity, and effectiveness of the administration mode for the patient, physician, nurse, or other caregiver), or other factors or combinations of these factors.

Conventional drug delivery technologies present various challenges. Oral administration of a dosage form is a relatively simple delivery mode, but some drugs may not achieve the desired bioavailability and/or may cause undesirable side effects if administered orally. Further, the delay from time of administration to time of efficacy associated with oral delivery may be undesirable depending on the therapeutic need. While parenteral administration by injection may avoid some of the problems associated with oral administration, such as providing relatively quick delivery of the drug to the desired location, conventional injections may be inconvenient, difficult to self-administer, and painful or unpleasant for the patient. Furthermore, injection may not be suitable for achieving certain delivery/release profiles, particularly over a sustained period of time.

Passive transdermal technology, such as a conventional transdermal patch, may be relatively convenient for the user and may permit relatively uniform drug release over time. However, some drugs, such as highly charged or polar drugs, peptides, proteins and other large molecule active agents, may not penetrate the stratum corneum for effective delivery. Furthermore, a relatively long start-up time may be required before the drug takes effect. Thereafter, the drug release may be relatively continuous, which may be undesirable in some cases. Also, a substantial portion of the drug payload may be undeliverable and may remain in the patch once the patch is removed.

Active transdermal systems, including iontophoresis, sonophoresis, and poration technology, may be expensive and may yield unpredictable results. Only some drug formulations, such as aqueous stable compounds, may be suited for active transdermal delivery. Further, modulating or controlling the delivery of drugs using such systems may not be possible without using complex systems.

Some infusion pump systems may be large and may require tubing between the pump and the infusion set, which can impact the quality of life of the patient. Further, infusion pumps may be expensive and may not be disposable. From the above, it would be desirable to provide new and improved drug delivery systems and methods that overcome some or all of these and other drawbacks.

### SUMMARY OF THE INVENTION

Devices and methods for delivering a fluid to a patient are disclosed herein. In one embodiment, a delivery system includes a reservoir configured to contain a fluid and a fluid communicator in fluid communication with the reservoir. An electrochemical actuator is coupled to the reservoir and configured to exert a force on the reservoir upon actuation such that fluid within the reservoir is communicated through the fluid communicator. The actuator includes a first end that is constrained and a second end that is not constrained. The actuator is configured to bend at a location along a length of the actuator when actuated such that the second end of the actuator is displaced in a direction toward the fluid reservoir. The actuator can be an electrochemical actuator. The apparatus can further include a transfer structure disposed between the actuator and the reservoir configured to contact the reservoir upon actuation of the actuator.

### BRIEF DESCRIPTION OF THE DRAWINGS

FIG. 1 is a schematic illustration of a delivery system according to an embodiment.

FIG. 2A is a side view of a schematic illustration of an electrochemical actuator shown in a charged state; and FIG. 2B is a schematic illustration of a side view of the electrochemical actuator of FIG. 2A shown in a discharged state.

FIG. 3A is a schematic illustration of a portion of a delivery system according to an embodiment illustrating an electrochemical actuator in a charged state; and FIG. 3B is a schematic illustration of the portion of the delivery system of FIG. 3A illustrating the electrochemical actuator as it discharges.

FIG. 3C is a schematic illustration of a portion of a delivery system according to an embodiment illustrating an electrical circuit including a first electrochemical actuator and a second electrochemical actuator.

FIG. 4A is a perspective view of a delivery system according to an embodiment and FIG. 4B is an exploded view of the delivery system of FIG. 4A.

FIG. 5A is a schematic illustration showing a mode of operation of an unclamped electrochemical actuator; and FIG. 5B is a schematic illustration showing a mode of operation of the electrochemical actuator of FIG. 5A with one end clamped.

FIG. 6A is a schematic illustration showing a mode of operation of an unclamped electrochemical actuator; and FIG. 6B is a schematic illustration showing a mode of operation of the electrochemical actuator of FIG. 6A with one end clamped.

FIG. 7A is a schematic illustration of an embodiment of an electrochemical actuator shown in a first configuration; and FIG. 7B is a schematic illustration of the electrochemical actuator of FIG. 7A shown in a second configuration.

FIG. 8 is a perspective view of a portion of a delivery device according to another embodiment.

FIG. 9 is a top view of a portion of the delivery device shown in FIG. 8 with a top portion of the housing removed.

FIG. 10 is a top view of a portion of the delivery device shown in FIG. 8 with a top portion of the housing and the transfer structure removed.

FIG. 11 is a perspective view of a fluid reservoir of the delivery device of FIG. 8.

FIGS. 12-14 are side views of a portion of the delivery device of FIG. 8 with a side wall of the housing removed and shown in first, second, and third configurations, respectively.

FIG. 15 is a perspective view of a portion of a delivery device according to another embodiment.

FIG. 16 is a top view of a portion of the delivery device shown in FIG. 15 with a top portion of the housing removed.

FIG. 17 is a top view of a portion of the delivery device shown in FIG. 15 with a top portion of the housing and the transfer structure removed.

FIG. 18 is a perspective view of a fluid reservoir of the delivery device of FIG. 15.

FIGS. 19-21 are side views of a portion of the delivery device of FIG. 15 with a side wall of the housing removed and shown in first, second, and third configurations, respectively.

FIG. 22 is a top view of a portion of delivery device according to another embodiment.

FIGS. 23 and 24 are side views of the delivery device of FIG. 22 with a side wall of the housing removed showing the delivery device in first and second configurations, respectively.

FIGS. 25-29 are each a side view of a schematic illustration of a different embodiment of a delivery device, shown with a side wall of the housing removed.

FIGS. 30-32 are each a graph illustrating the results of testing of an electrochemical actuator clamped or held at different locations on the electrochemical actuator.

### DETAILED DESCRIPTION

Devices, systems and methods are described herein that are configured for use in the delivery of therapeutic agents to a patient's body. Such therapeutic agents can be, for example, one or more drugs and can be in fluid form of various viscosities. In some embodiments, the devices and methods can include a pump device that includes an actuator, such as, for example, an electrochemical actuator, which can have characteristics of both a battery and a pump. Specifically, an electrochemical actuator can include an electrochemical cell that produces a pumping force as the cell discharges. Thus, the pump device can have relatively fewer parts than a conventional drug pump, such that the pump device is relatively more compact, disposable, and reliable than conventional drug pumps. Such drug delivery devices are desirable, for example, for use in delivery devices that are designed to be attached to a patient's body (e.g., a wearable device). These attributes of the pump device may reduce the cost and the discomfort associated with infusion drug therapy.

In some embodiments, such a pump device can be operated with, for example, a controller and/or other circuitry, operative to regulate drug or fluid flow from the pump device. Such a controller may permit implementing one or more release profiles using the pump device, including release profiles that require uniform flow, non-uniform flow, continuous flow, discontinuous flow, programmed flow, scheduled flow, user-initiated flow, or feedback responsive flow, among others. Thus, the pump device may effectively deliver a wider variety of drug therapies than other pump devices.

The systems and methods described herein can include an electrochemical actuator, such as a self-powered actuator and/or combined battery and actuator. Example embodiments of such electrochemical actuators are generally described in U.S. Pat. No. 7,541,715, entitled “Electrochemical Methods, Devices, and Structures” by Chiang et al., U.S. Patent Pub. No. 2008/0257718, entitled “Electrochemical Actuator” by Chiang et al., and U.S. Patent Pub. No. 2009/0014320, entitled “Electrochemical Actuator” by Chiang et al., and U.S. Pat. No. 7,828,771, entitled “Systems and Methods for Delivering Drugs” by Chiang et al., (the '771 patent), the disclosure of each of which is incorporated herein by reference. Such electrochemical actuators can include at least one component that responds to the application of a voltage or current by experiencing a change in volume or position. The change in volume or position can produce mechanical work that can then act on a fluid source (e.g., fluid reservoir 104) or may be transferred to a fluid source, such that a fluid can be delivered out of the fluid source.

In some embodiments of a delivery system, an electrochemical actuator is configured as an elongate plate, which bends when actuated. The actuator can be clamped or otherwise constrained at one end, so that the actuator is cantilevered from that end. In such an arrangement an increased range of motion at the free end for the same angular deflection of the electrochemical actuator can be achieved and/or an increased rate of actuation for the same vertical tip deflection. In some cases, an increased range of motion and/or an increased actuation rate can also result in a reduction in the tip force that it can apply to pump fluid out of a fluid reservoir. This change in force may be dependent on the externally applied load that affects the stress at the clamp location. In some embodiments, however, such a reduction in tip force can be tolerated. In some embodiments, a delivery device having an electrochemical actuator with one end clamped, can result in approximately doubling of the vertical deflection of the actuator. Thus, the useful stroke of the actuator can be effectively doubled (depending on angular actuator displacement). In general, the size of the actuator, coupled with the location of the push (e.g., location where the actuator pushes on a transfer structure and/or fluid source as described herein) and cantilever points can be leveraged to change the interplay of vertical displacement and available force. With the same actuator, moving the push point closer to the pivot will act to increase the piston's displacement rate but also increase the force requirements on the actuator. Conversely, moving the push point away from the pivot will act to decrease force requirements (thus enabling the use of weaker, possibly cheaper actuators) but also reduce displacement rate, and require generally larger vertical stroke from the actuator.

Electrochemical actuators can provide volume-efficient capabilities that are especially effective in applications where minimal weight and volume are desired. Example applications are those of drug/medication patch pumps that are worn by a patient. While most pumps use a variety of prime movers that either require external drive circuitry or power, bulky, expensive, and/or complex, electrochemical actuator-based pumps have significant advantages by virtue of having a small actuator volume and no need for an external power source.

By clamping an end of an electrochemical actuator used in a drug delivery device, the device and/or actuator can be asymmetric, thus further saving both volume and material (and therefore cost). In some embodiments of a drug delivery device, an electrochemical actuator can include rigid external legs coupled to one end or opposite ends of the actuator. A rigid leg can be used as an interface between the actuator and the clamping mechanism and can also house suitable drive electronics (from the simplest version of a discharge resistor and an activation switch to more complex communication units). This additional configuration can further optimize features of the basic electrochemical actuator (such as minimal size that can sustain the load, reduced complexity and cost, simple fabrication, etc.) and leave interfacing with loads and the package to the external legs. The electronics can include some or all of the necessary drive circuitry, communication units, as well as a switch to activate motion as needed.

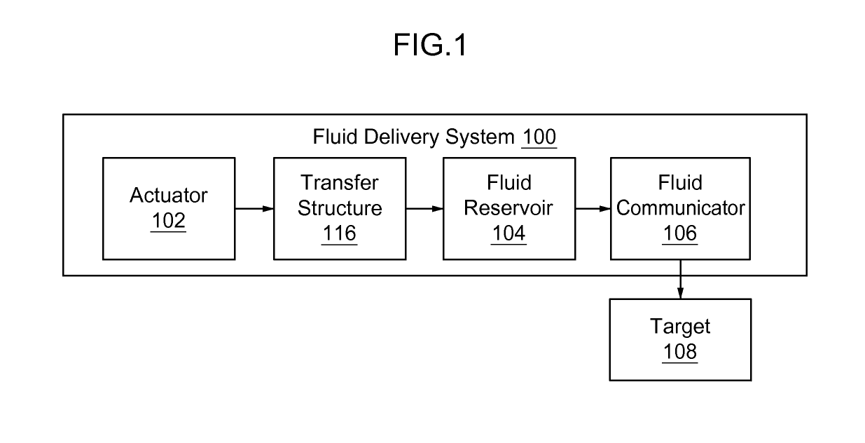

FIG. 1 is a schematic block diagram illustrating an embodiment of a fluid delivery system 100 (also referred to herein as “delivery device” or “drug delivery device”). The fluid delivery system 100 includes an actuator 102, a transfer structure 116, a fluid source 104 and a fluid communicator 106. The fluid source 104 can contain a fluid (i.e., a therapeutic agent) to be delivered into a target 108 via the fluid communicator 106. The target 108 can be, for example, a human or other mammalian body in need of a drug therapy or prophylaxis.

The actuator 102 can be, for example, an electrochemical actuator 102 that can actuate or otherwise create a pumping force to deliver the fluid from the fluid source 104 into the fluid communicator 106 as described in more detail below. In some embodiments, the actuator 102 can be a device that experiences a change in volume or position in response to an electrochemical reaction that occurs therein. For example, the actuator 102 can be an electrochemical actuator that includes a charged electrochemical cell, and at least a portion of the electrochemical cell can actuate as the electrochemical cell discharges. Thus, the actuator 102 can be considered a self-powered actuator or a combination battery and actuator.

The fluid source 104 can be a reservoir, pouch, chamber, barrel, bladder, or other known device that can contain a drug in fluid form therein. The fluid communicator 106 can be in, or can be moved into, fluid communication with the fluid source 104. The fluid communicator 106 can be, for example, a needle, catheter, cannula, infusion set, or other known drug delivery conduit that can be inserted into or otherwise associated with the target body for drug delivery.

In some embodiments, the fluid source 104 can be any component capable of retaining a fluid or drug in fluid form. In some embodiments, the fluid source 104 may be disposable (e.g., not intended to be refillable or reusable). In other embodiments, the fluid source 104 can be refilled, which may permit reusing at least a portion of the device and/or varying the drug or fluid delivered by the device. In some embodiments, the fluid source 104 can be sized to correlate with the electrochemical potential of the electrochemical actuator 102. For example, the size and/or volume of the fluid source 104 can be selected so that the fluid source 104 becomes about substantially empty at about the same time that the electrochemical actuator 102 becomes about substantially discharged. By optimizing the size of the fluid source 104 and the amount of drug contained therein to correspond to the driving potential of the electrochemical actuator 102, the size and/or cost of the device may be reduced. In other embodiments, the electrochemical actuator 102 may be oversized with reference to the fluid source 104. In some embodiments, the delivery system 100 can include more than one fluid source 104. Such a configuration may permit using a single device to deliver two or more drugs or fluids. The two or more drugs or fluids can be delivered discretely, simultaneously, alternating, according to a program or schedule, or in any other suitable manner. In such embodiments, the fluid sources 104 may be associated with the same or different electrochemical actuators 102, the same or different fluid communicators 106, the same or different operational electronics, or the same or different portions of other components of the delivery system.

The transfer structure 116 can be disposed between the electrochemical actuator 102 and the fluid source 104. The transfer structure 116 includes a surface configured to contact the fluid source 104 upon actuation of the actuator 102 such that a force exerted by the electrochemical actuator 102 is transferred from the transfer structure 116 to the fluid source 104. The transfer structure 116 can include one or more components. For example, the transfer structure 116 can be a single component having a surface configured to contact the fluid source 104. In some embodiments, the transfer structure 116 can include one or more members having a surface configured to contact the fluid source 104 upon activation of the electrochemical actuator 102. In some embodiments, the transfer structure 116 is a substantially planar or flat plate.

The actuator 102 can be fixed at one end, e.g. by coupling to a clamping mechanism (not shown in FIG. 1) and the opposite end can be unconstrained. With one end fixed, the actuator 102 can deflect or bend when activated such that the free end bends or rotates about a pivot location as described in more detail below with reference to specific embodiments. The transfer structure 116 can be pivotally coupled at one end to a mounting member (not shown in FIG. 1) and include a free end at an opposite end. Upon activation of the actuator 102, the transfer structure 116 can pivot about its pivot coupling. For example, as the actuator 102 is activated and begins to bend or deflect, the portion of the actuator 102 can contact and exert a force on the transfer structure 116 and cause the transfer structure 116 to move or rotate about its pivotal mounting location. As the transfer structure 116 moves, it can contact the fluid source 104 as described above, to cause the fluid within the fluid source 104 to be discharged out of the fluid source 104 and into the patient. It is also possible to clamp the actuator 102 to the transfer structure 116 and push against the bottom of the housing. This may be a desirable configuration to optimize how the system is supplied and/or assembled/activated by the end user.

In some embodiments, the fluid delivery system 100 can be used to deliver a drug formulation which comprises a drug, including an active pharmaceutical ingredient. In other embodiments, the fluid delivery system 100 may deliver a fluid that does not contain a drug. For example, the fluid may be a saline solution or a diagnostic agent, such as a contrast agent. Drug delivery can be subcutaneous, intravenous, intraarterial, intramuscular, intracardiac, intraosseous, intradermal, intrathecal, intraperitoneal, intratumoral, intratympnic, intraaural, topical, epidural, and/or peri-neural depending on, for example, the location of the fluid communicator 106 and/or the entry location of the drug.

The drug (also referred to herein as “a therapeutic agent” or “a prophylactic agent”) can be in a pure form or formulated in a solution, a suspension, or an emulsion, among others, using one or more pharmaceutically acceptable excipients known in the art. For example, a pharmaceutically acceptable vehicle for the drug can be provided, which can be any aqueous or non-aqueous vehicle known in the art. Examples of aqueous vehicles include physiological saline solutions, solutions of sugars such as dextrose or mannitol, and pharmaceutically acceptable buffered solutions, and examples of non-aqueous vehicles include fixed vegetable oils, glycerin, polyethylene glycols, alcohols, and ethyl oleate. The vehicle may further include antibacterial preservatives, antioxidants, tonicity agents, buffers, stabilizers, or other components.

Although the fluid delivery system 100 and other systems and methods described herein are generally described as communicating drugs into a human body, such systems and methods may be employed to deliver any fluid of any suitable biocompatibility or viscosity into any object, living or inanimate. For example, the systems and methods may be employed to deliver other biocompatible fluids into living beings, including human beings and other animals. Further, the systems and methods may deliver drugs or other fluids into living organisms other than human beings, such as animals and plant life. Also, the systems and methods may deliver any fluids into any target, living or inanimate.

In some embodiments, the electrochemical actuator 102 can include a positive electrode and a negative electrode, at least one of which is an actuating electrode. These and other components of the electrochemical actuator can form an electrochemical cell, which can in some embodiments initially be charged. For example, the electrochemical cell may begin discharging when a circuit between the electrodes is closed, causing the actuating electrode to actuate. The actuating electrode can thereby perform work upon another structure, such as the fluid source, or a transfer structure associated with the fluid source, as described in more detail below. The work can then cause fluid to be pumped or otherwise dispensed from the fluid source into the target 108.

More specifically, the actuating electrode of the electrochemical actuator 102 can experience a change in volume or position when the closed circuit is formed, and this change in volume or position can perform work upon the fluid source or transferring structure. For example, the actuating electrode may expand, bend, buckle, fold, cup, elongate, contract, or otherwise experience a change in volume, size, shape, orientation, arrangement, or location, such that at least a portion of the actuating electrode experiences a change in volume or position. In some embodiments, the change in volume or position may be experienced by a portion of the actuating electrode, while the actuating electrode as a whole may experience a contrary change or no change whatsoever. It is noted that the delivery device 100 can include more than one electrochemical actuator 102. For example, in some embodiments, the delivery device 100 can include one or more electrochemical actuators 102 arranged in series, parallel, or some combination thereof. In some embodiments, a number of such electrochemical actuators 102 may be stacked together. As another example, concurrent or sequenced delivery of multiple agents can be achieved by including one or more electrochemical actuators 102 acting on two or more fluid sources.

The delivery system 100 can also include a housing (not shown in FIG. 1) that can be removably or releasably attached to the body (e.g., the skin) of the patient. The various components of the delivery system 100 can be fixedly or releasably coupled to the housing. For example, the clamping mechanism and the mounting member described above can be formed integrally with a portion of the housing, or can be coupled to the housing.

To adhere the delivery device 100 to the skin of a patient, a releasable adhesive can at least partially coat an underside of the housing. The adhesive can be non-toxic, biocompatible, and releasable from human skin. To protect the adhesive until the device is ready for use, a removable protective covering can cover the adhesive, in which case the covering can be removed before the device is applied to the skin. Alternatively, the adhesive can be heat or pressure sensitive, in which case the adhesive can be activated once the device is applied to the skin. Example adhesives include, but are not limited to, acrylate based medical adhesives of the type commonly used to affix medical devices such as bandages to skin. However, the adhesive is not necessary, and may be omitted, in which case the housing can be associated with the skin, or generally with the body, in any other manner. For example, a strap or band can be used.

The housing can be formed from a material that is relatively lightweight and flexible, yet sturdy. The housing also can be formed from a combination of materials such as to provide specific portions that are rigid and specific portions that are flexible. Example materials include plastic and rubber materials, such as polystyrene, polybutene, carbonate, urethane rubbers, butene rubbers, silicone, and other comparable materials and mixtures thereof, or a combination of these materials or any other suitable material can be used.

In some embodiments, the housing can include a single component or multiple components. In some embodiments, the housing can include two portions: a base portion and a movable portion. The base portion can be suited for attaching to the skin. For example, the base portion can be relatively flexible. An adhesive can be deposited on an underside of the base portion, which can be relatively flat or shaped to conform to the shape of a particular body part or area. The movable portion can be sized and shaped for association with the base portion. In some embodiments, the two portions can be designed to lock together, such as via a locking mechanism. In some cases, the two portions can releasably lock together, such as via a releasable locking mechanism, so that the movable portion can be removably associated with the base portion. To assemble such a housing, the movable portion can be movable with reference to the base portion between an unassembled position and an assembled position. In the assembled position, the two portions can form a device having an outer shape suited for concealing the device under clothing. Various example embodiments of a housing are described in the '771 patent.

The size, shape, and weight of the delivery device 100 can be selected so that the delivery device 100 can be comfortably worn on the skin after the device is applied via the adhesive. For example, the delivery device 100 can have a size, for example, in the range of about 1.0″×1.0″×0.1″ to about 5.0″×5.0″×1.0″, and in some embodiments in a range of about 2.0″×2.0″×0.25″ to about 4.0″×4.0″×0.67″. The weight of the delivery device 100 can be, for example, in the range of about 5 g to about 200 g, and in some embodiments in a range of about 15 g to about 100 g. The delivery device 100 can be configured to dispense a volume in the range of about 0.1 ml to about 1,000 ml, and in some cases in the range of about 0.3 ml to about 100 ml, such as between about 0.5 ml and about 5 ml. The shape of the delivery device can be selected so that the delivery device 100 can be relatively imperceptible under clothing. For example, the housing can be relatively smooth and free from sharp edges. However, other sizes, shapes, and/or weights are possible.

As mentioned above, an electrochemical actuator 102 can be used to cause the fluid delivery device 100 to deliver a drug-containing or non-drug containing fluid into a human patient or other target 108. Such a fluid delivery system 100 can be embodied in a relatively small, self-contained, and disposable device, such as a patch device that can be removably attached to the skin of patient as described above. The delivery device 100 can be relatively small and self-contained, in part, because the electrochemical actuator 102 serves as both the battery and a pump. The small and self-contained nature of the delivery device 100 advantageously may permit concealing the device beneath clothing and may allow the patient to continue normal activity as the drug is delivered. Unlike conventional drug pumps, external tubing to communicate fluid from the fluid reservoir into the body can be eliminated. Such tubing can instead be contained within the delivery device, and a needle or other fluid communicator can extend from the device into the body. The electrochemical actuator 102 can initially be charged, and can begin discharging once the delivery device 100 is activated to pump or otherwise deliver the drug or other fluid into the target 108. Once the electrochemical actuator 102 has completely discharged or the fluid source 104 (e.g. reservoir) is empty, the delivery device 100 can be removed. The small and inexpensive nature of the electrochemical actuator 102 and other components of the device may, in some embodiments, permit disposing of the entire delivery device 100 after a single use. The delivery device 100 can permit drug delivery, such as subcutaneous or intravenous drug delivery, over a time period that can vary from several minutes to several days. Subsequently, the delivery device 100 can be removed from the body and discarded.

In use, the delivery device 100 can be placed in contact with the target 108 (e.g. placed on the surface of a patient's body), such that the fluid communicator 106 (e.g., a needle, cannula, etc.) is disposed adjacent to a desired injection site. The fluid communicator 106 can be actuated with the actuation of the electrochemical actuator 102 or separately as described in more detail below. For example, the delivery device 100 can include a separate mechanism to actuate the fluid communicator 106. Activation of the fluid communicator 106 can include, for example, insertion of the fluid communicator 106 into the patient's body. Example embodiments illustrating various configurations for actuation of the fluid communicator 106 are described in the '771 patent incorporated by reference above. The electrochemical actuator 102 can then be actuated to apply a force on the fluid source 104, causing the fluid to be delivered through the fluid communicator 106 and into the target 108. For example, as the electrochemical actuator 102 is actuated, the actuator 102 will be displaced and will contact and apply a force to the transfer structure 116 and that force will in turn be transferred to the fluid source 104 to pump the fluid out of the fluid source 104, through the fluid communicator 106, and into the target 108.

Having described above various general principles, several exemplary embodiments of these concepts are now described. These embodiments are only examples, and many other configurations of a delivery system and/or the various components of a delivery system, are contemplated.

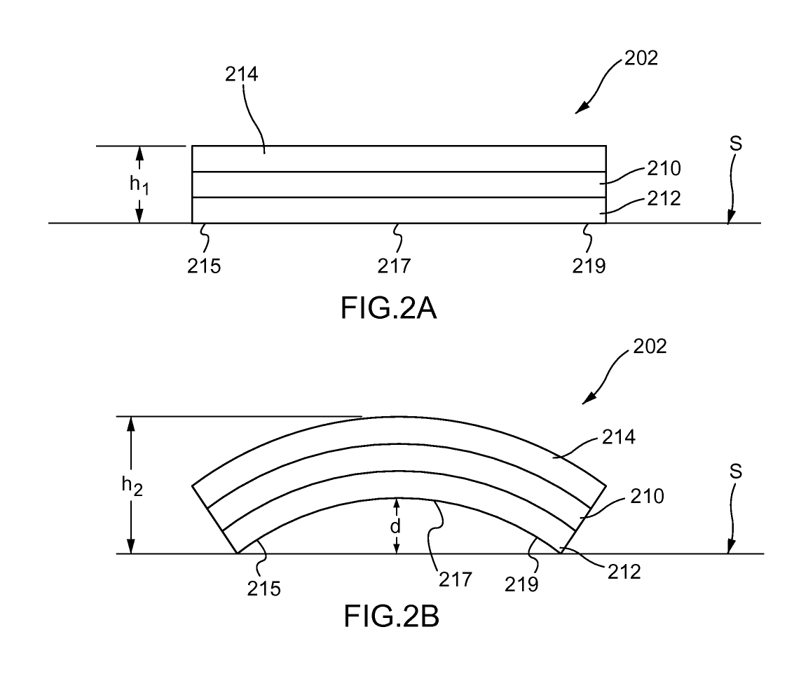

FIGS. 2A and 2B are schematic illustrations of an embodiment of an electrochemical actuator 202 that can be used in a delivery device as described herein. As shown, in this embodiment, the electrochemical actuator 202 can include a positive electrode 210, a negative electrode 212, and an electrolyte 214. These components can form an electrochemical cell that can initially be discharged and then charged before use, or can be initially charged, as shown in FIG. 2A. The positive electrode 210 can be configured to expand or displace in the presence of the electrolyte 214. When a circuit between the electrodes 210, 212 is closed, current can travel from the positive electrode 210 to the negative electrode 212. The positive electrode 210 can then experience a change in volume or shape, resulting in longitudinal displacement of at least a portion of the positive electrode 210, as shown in FIG. 2B. For example, the actuator 202 can have an overall height h₁ when it is charged (prior to actuation), as shown in FIG. 2A, and an overall height of h₂ when it is discharged or actuated, such that the actuator 202 has a displacement or stroke that is equal to h₂-h₁. Said another way, the actuator 202 can have a first end portion 215, a second end portion 219 and a medial portion 217 disposed between the first end portion 215 and the second end portion 219. The actuator prior to actuation (prior to discharge) can be supported on a surface S of the delivery device in which the actuator 202 is disposed, and when the actuator 202 is discharged at least the medial portion 217 can displace (e.g., bend or flex) a non-zero distance d from the surface S. The stroke of the actuator 202 can be substantially equal to that non-zero distance d. As the actuator 202 is displaced, the actuator 202 can exert a pumping force or pressure on a fluid reservoir (not shown) and/or an associated transfer structure (not shown) coupled thereto. The pumping force or pressure exerted by the actuator 202 can cause a volume of fluid (e.g., a therapeutic agent) to be pumped out of the fluid reservoir. Thus, the electrochemical actuator 202 can be considered a self-powered electrochemical pump.

In this embodiment, the electrochemical actuator 202 has a positive electrode 210 selected to have a lower chemical potential for the working ion when the electrochemical actuator 202 is charged, and is thereby able to spontaneously accept working ions from the negative electrode 212 as the actuator is discharged. In some embodiments, the working ion can include, but is not limited to, the proton or lithium ion. When the working ion is lithium, the positive electrode 210 can include one or more lithium metal oxides including, for example, LiCoO₂, LiFePO₄, LiNiO₂, LiMn₂O₄, LiMnO₂, LiMnPO₄, Li₄Ti₅O₁₂, and their modified compositions and solid solutions; oxide compound comprising one or more of titanium oxide, manganese oxide, vanadium oxide, tin oxide, antimony oxide, cobalt oxide, nickel oxide or iron oxide; metal sulfides comprising one or more of TiSi₂, MoSi₂, WSi₂, and their modified compositions and solid solutions; a metal, metal alloy, or intermetallic compound comprising one or more of aluminum, silver, gold, boron, bismuth, gallium, germanium, indium, lead, antimony, silicon, tin, or zinc; a lithium-metal alloy; or carbon comprising one or more of graphite, a carbon fiber structure, a glassy carbon structure, a highly oriented pyrolytic graphite, or a disordered carbon structure. The negative electrode 212 can include, for example, lithium metal, a lithium metal alloy, or any of the preceding compounds listed as positive electrode compounds, provided that such compounds when used as a negative electrode are paired with a positive electrode that is able to spontaneously accept lithium from the negative electrode when the actuator is charged. These are just some examples, as other configurations are also possible.

In some embodiments, the electrochemical actuator can include an anode, a cathode, and a species, such as a lithium ion. In some embodiments, a source of lithium ion is the electrolyte which is made up an organic solvent such as PC, propylene carbonate, GBL, gamma butyl lactone, dioxylane, and others, and an added electrolyte. Some example electrolytes include LiPF₆, LiBr, LiBF₄. At least one of the electrodes can be an actuating electrode that includes a first portion and a second portion. The portions can have at least one differing characteristic, such that in the presence of a voltage or current, the first portion responds to the species in a different manner than the second portion. For example, the portions can be formed from different materials, or the portions can differ in thickness, dimension, porosity, density, or surface structure, among others. The electrodes can be charged, and when the circuit is closed, current can travel. The species can, intercalate, de-intercalate, alloy with, oxide, reduce, or plate with the first portion to a different extent than the second portion. Due to the first portion responding differently to the species than the second portion, the actuating electrode can experience a change in one or more dimensions, volume, shape, orientation, or position.

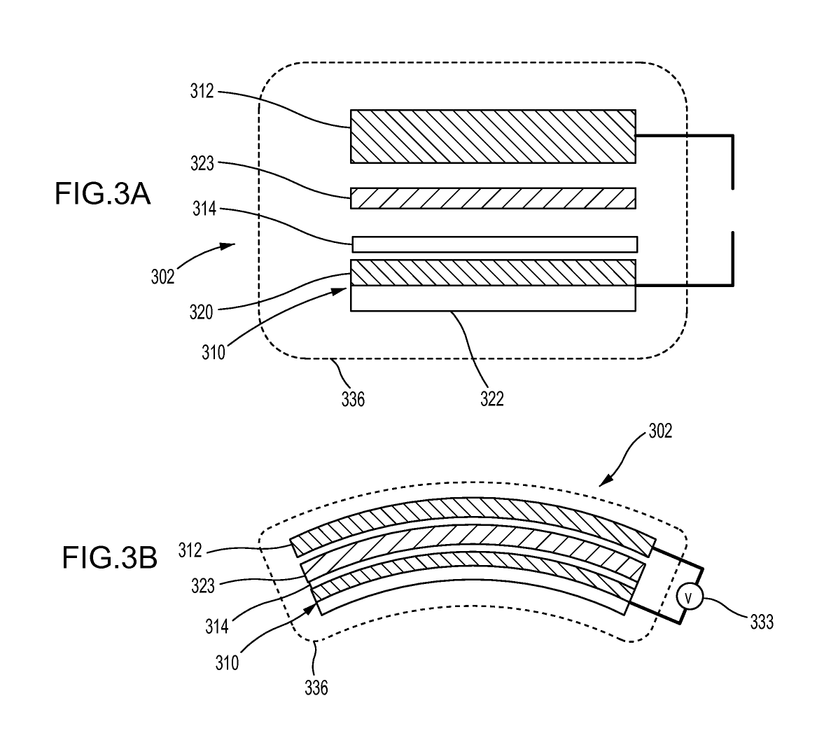

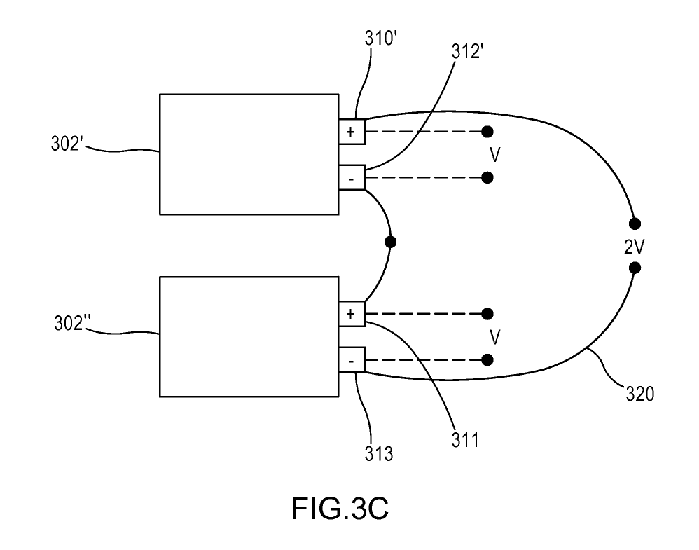

Another example of an electrochemical actuator is shown in the embodiment illustrated in FIGS. 3A and 3B. As shown in FIG. 3A, an electrochemical actuator 302 can include a negative electrode 312 in electrical communication with a positive electrode 310 collectively forming an electrochemical cell. Positive electrode 310 may include a first portion 320 and a second portion 322. In some embodiments, first portion 320 and second portion 322 are formed of different materials. Portions 320 and 322 may also have different electrical potentials. For example, first portion 320 may include a material that can intercalate, de-intercalate, alloy with, oxidize, reduce, or plate a species to a different extent than second portion 322. Second portion 322 may be formed of a material that does not substantially intercalate, de-intercalate, or alloy with, oxidize, reduce, or plate the species. In some embodiments, first portion 320 may be formed of a material including one or more of aluminum, antimony, bismuth, carbon, gallium, silicon, silver, tin, zinc, or other materials which can expand upon intercalation or alloying or compound formation with lithium. In one embodiment, first portion 320 is formed with aluminum, which can expand upon intercalation with lithium. Second portion 322 may be formed of copper, since copper does not substantially intercalate or alloy with lithium. In some instances, second portion 322 may act as a positive electrode current collector, and may extend outside the electrochemical cell, e.g., to form a tab or current lead. In other embodiments, second portion 322 may be joined to a tab or current lead that extends outside the cell. Negative electrode 312 may also include a current collector. Electrochemical actuator 302 may include a separator 323. The separator 323 may be, for example, a porous separator film, such as a glass fiber cloth, or a porous polymer separator. Other types of separators, such as those used in the construction of lithium ion batteries, may also be used. The electrochemical actuator 302 may also include an electrolyte 314, which may be in the form of a liquid, solid, or a gel. The electrolyte may contain an electrochemically active species, such as that used to form the negative electrode. Electrochemical actuator 302 may also include an enclosure 336, such as a polymer packaging, in which negative electrode 312, positive electrode 310 and separator 323 can be disposed.

As illustrated in FIG. 3B, the electrochemical cell may have a voltage 333, such that, when a closed circuit is formed between the negative electrode 312 and the positive electrode 310, an electric current may flow between the negative electrode 312 and the positive electrode 310 through the external circuit. If negative electrode 312 is a lithium metal electrode and the electrolyte contains lithium ions, lithium ion current can flow internally from the negative electrode 312 to the positive electrode 310. The intercalation of first portion 320 with lithium can result in a dimensional change, such as a volume expansion. In some instances, this volume expansion may reach at least 25%, at least 50%, at least 75%, at least 100%, at least 150%, at least 200%, at least 250%, or at least 300% compared to the initial volume. High volume expansion may occur, for example, when first portion 320 is saturated with lithium. As first portion 320 increases in volume due to intercalation of lithium, second portion 322 to which first portion 320 may be bonded, may not substantially expand due to minimal or no intercalation of lithium. First portion 320 thus provides a mechanical constraint. This differential strain between the two portions causes positive electrode 310 to undergo bending or flexure. As a result of the dimensional change and displacement of the positive electrode 310, electrochemical actuator 302 can be displaced from a first orientation to a second orientation. This displacement can occur whether the volumetric or dimensional change (e.g., net volume change) of the electrochemical cell, due to the loss of lithium metal from the negative electrode 312 and formation of lithium intercalated compound or lithium alloy at the positive electrode 310, is positive, zero, or negative. In some cases, the actuator displacement may occur with a volumetric or dimensional change (e.g., net volume change) of the electrochemical actuator 302, or portion thereof that is positive. In some cases, the actuator displacement may occur with a volumetric or dimensional change (e.g., net volume change) of the electrochemical actuator 302, or portion thereof that is zero. In some cases, the actuator displacement may occur with a volumetric or dimensional change (e.g., net volume change) of the electrochemical actuator 302, or portion thereof that is negative.

As used herein, “differential strain” between two portions can refer to the difference in response (e.g., actuation) of each individual portion upon application of a voltage or current to the two portions. That is, a system as described herein may include a component including a first portion and a second portion associated with (e.g., may contact, may be integrally connected to) the first portion, wherein, under essentially identical conditions, the first portion may undergo a volumetric or dimensional change and the second portion does not undergo a volumetric or dimensional change, producing strain between the first and second portions. The differential strain may cause the component, or a portion thereof, to be displaced from a first orientation to a second orientation. In some embodiments, the differential strain may be produced by differential intercalation, de-intercalation, alloying, oxidation, reduction, or plating of a species with one or more portions of the actuator system.

For example, the differential intercalation, de-intercalation, alloying, oxidation, reduction, or plating of first portion 320 relative to second portion 322 can be accomplished through several means. In one embodiment, first portion 320 may be formed of a different material than second portion 322, wherein one of the materials substantially intercalates, de-intercalates, alloys with, oxidizes, reduces, or plates a species, while the second portion interacts with the species to a lesser extent. In another embodiment, first portion 320 and second portion 322 may be formed of the same material. For example, first portion 320 and second portion 322 may be formed of the same material and may be substantially dense, or porous, such as a pressed or sintered powder or foam structure. In some cases, to produce a differential strain upon operation of the electrochemical cell, first portion 320 or second portion 322 may have sufficient thickness such that, during operation of the electrochemical cell, a gradient in composition may arise due to limited ion transport, producing a differential strain. In some embodiments, one portion or an area of one portion may be preferentially exposed to the species relative to the second portion or area of the second portion. In other instances, shielding or masking of one portion relative to the other portion can result in lesser or greater intercalation, de-intercalation, or alloying with the masked or shielded portion compared to the non-masked or shielded portion. This may be accomplished, for example, by a surface treatment or a deposited barrier layer, lamination with a barrier layer material, or chemically or thermally treating the surface of the portion to be masked/shielded to either facilitate or inhibit intercalation, de-intercalation, alloying, oxidation, reduction, or plating with the portion. Barrier layers can be formed of any suitable material, which may include polymers, metals, or ceramics. In some cases, the barrier layer can also serve another function in the electrochemical cell, such as being a current collector. The barrier layer may be uniformly deposited onto the surface in some embodiments. In other cases, the barrier layer may form a gradient in composition and/or dimension such that only certain portions of the surface preferentially facilitate or inhibit intercalation, de-intercalation, alloying, oxidation, reduction, or plating of the surface. Linear, step, exponential, and other gradients are possible. In some embodiments a variation in the porosity across first portion 320 or second portion 322, including the preparation of a dense surface layer, may be used to assist in the creation of an ion concentration gradient and differential strain. Other methods of interaction of a species with a first portion to a different extent so as to induce a differential strain between the first and second portions can also be used. In some embodiments, the flexure or bending of an electrode is used to exert a force or to carry out a displacement that accomplishes useful function.

In some embodiments, the electrical circuit can include electrical contacts (not shown) that can open or close the electrical circuit. For example, when the electrical contacts are in communication with each other, the electrical circuit will be closed (as shown in FIG. 3B) and when they are not in contact with each other, the electrical circuit can be opened or broken, as shown in FIG. 3A.

The discharge of the electrochemical actuator can be relatively proportional to the current traveling through the electrical circuit (i.e., the electrical resistance of the resistor). Because the electrical resistance of the resistor can be relatively constant, the electrochemical actuator can discharge at a relatively constant rate. Thus, the discharge of the electrochemical actuator, and thus the displacement of the electrochemical actuator can be relatively linear with the passage of time.

In some embodiments, an electrical circuit can be used that includes a variable resistor. By varying the resistance, the discharge rate of the electrochemical actuator and the corresponding displacement of the electrochemical actuator can be varied, which in turn can vary the fluid flow rate from the fluid source. An example of such an embodiment is described in the '771 patent. In some embodiments, an electrical circuit can be used that uses a switch to open or close the electrical circuit. When the switch is closed, the electrochemical actuator can discharge and when the switch is opened, the electrochemical actuator can be prevented from discharging. An example of such an embodiment is described in the '771 patent incorporated by reference above.

Although the foregoing discussion describes an electrical circuit formed between electrodes (e.g., 310, 312) of a single electrochemical actuator 302, in some embodiments, an electrical circuit can be formed between electrodes of multiple electrochemical actuators. For example, as schematically illustrated in FIG. 3C, an electrical circuit 320 can be used that includes a first electrochemical actuator 302′ and a second electrochemical actuator 302″. Each of the electrochemical actuators 302′, 302″ can be similar in many respects to electrochemical actuator 302 described above, except as noted herein.

Specifically, a positive electrode 310′ of the first actuator 302′ is in electrical communication with a negative electrode 313 of the second actuator 302″, and a negative electrode 312′ of the first actuator 302′ is in electrical communication with a positive electrode 311 of the second actuator 330. As such, whereas the electrochemical cell described above with reference to FIGS. 3A and 3B has a voltage 333 when a closed circuit is formed between its negative electrode 312 and its positive electrode 310, when a closed circuit is formed between the negative electrode 312′ of the first electrochemical actuator 302 and the positive electrode 311 of the second electrochemical actuator 302″ and between the negative electrode 313 of the second electrochemical actuator 302″ and the positive electrode 310′ of the first electrochemical actuator 302, as in the embodiment of FIG. 3C, a combined voltage 2V substantially equal to at least the sum of the voltage potential V of each electrochemical actuator 302′, 302″ is produced.

For example, if each electrochemical actuator 302′, 302″ has a voltage potential V substantially equal to the voltage 333 of the electrochemical cell described above, when the electrical circuit 320 is closed between the electrodes of the electrochemical actuators 302′, 302″, the electrical circuit has a voltage of about two times voltage 333. In another example, the first electrochemical actuator 302′ can have a voltage V of about 0.3 and the second electrochemical actuator 330 can have a voltage V of about 0.3. Because the first and second electrochemical actuators 302′, 302″ are included in the single (or same) electrical circuit 320, the effective or total voltage 2V of the circuit is about 0.6. In this manner, the displacement of each of the first and second electrochemical actuators 302′, 302″ can be greater in the presence of the total voltage 2V of the electrical circuit 320, for example, than would otherwise occur in the presence of the voltage V (e.g., an electrical circuit with a single actuator). Additionally, the electrochemical actuators 302′, 302″ can collectively produce sufficient power to drive electronic components of a delivery system which a single electrochemical actuator may have insufficient power to drive.

Although the electrochemical actuators 302′, 302′ are described as being about 0.3 volts individually, and 0.6 volts collectively, in other embodiments, each electrochemical actuator 302′, 302″ can have any suitable voltage. Furthermore, the electrochemical actuators 302′, 302″ can have the same voltage, or different voltages. Although the circuit 320 has been illustrated and described as including two electrochemical actuators 302′, 302″, in other embodiments, an electrical circuit can include three or more electrochemical actuators. Additionally, the electrochemical actuators 302′, 330 can be connected in parallel, effectively doubling the capacity (amp hours) of the electrochemical actuators 302′, 330 while maintaining the voltage of the electrical circuit at that of a single electrochemical actuator.

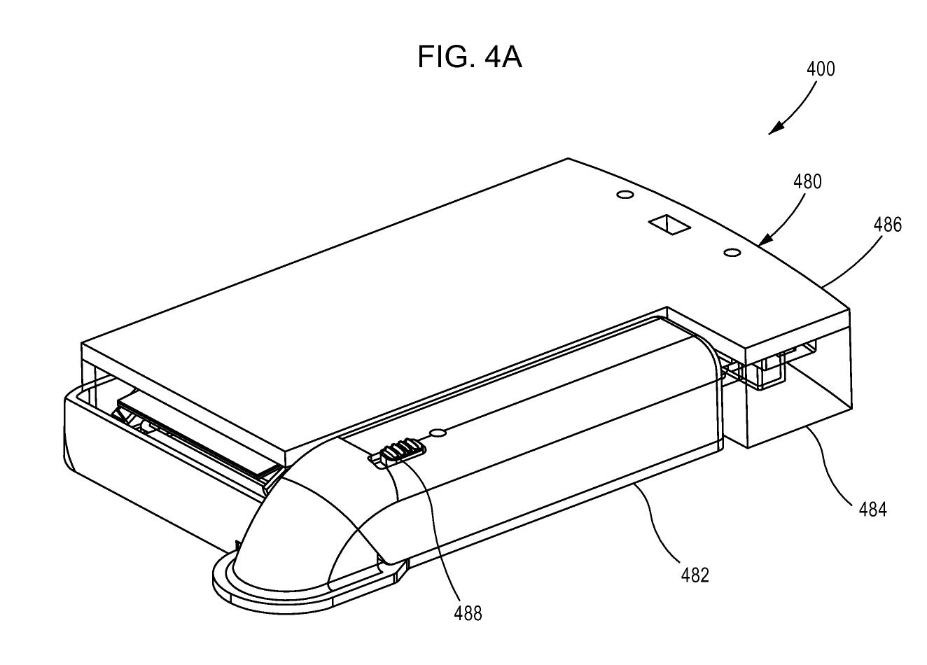

FIGS. 4A and 4B illustrate an embodiment of a delivery device that can include an electrochemical actuator as described herein. A delivery device 400 includes a housing 480, a fluid source 404, an electrochemical actuator 402, a transfer structure 416 disposed between the fluid source 404 and the actuator 402, a support member 485, and associated electronics (not shown) that can be coupled to the electrochemical actuator 402. In this embodiment, the housing 480 includes a first portion 482, a second portion 484, and a top portion 486 that can be coupled together to form an interior region within the housing 480. The fluid source 404, the electrochemical actuator 402, the support structure 485 and the transfer structure 416 can each be disposed within the interior region defined by the housing 480. The transfer structure 416 can be pivotally coupled to a mounting portion 438 on the support structure 485 via pivots 434. When assembled, an end portion of the actuator 402 is constrained with a clamping mechanism 430 of the support structure 485.

The fluid source 404 can be provided to a user predisposed within the interior region of the housing 480 or can be provided as a separate component that the user can insert into the housing 480. For example, the fluid source 404 can be inserted through an opening (not shown) in the housing 480. The fluid source 404 can be, for example, a fluid reservoir, bag or container, etc. that defines an interior volume that can contain a fluid to be injected into a patient. The fluid source 404 (also referred to herein as “fluid reservoir”) can include a web portion (not shown) configured to be punctured by an insertion mechanism (not shown) to create a fluid channel between the fluid source 404 and a fluid communicator (not shown) configured to penetrate the patient's skin. In some embodiments, the fluid reservoir 404 can be sized for example, with a length L of about 2 cm, a width W of about 2 cm, and a height H of about 0.25 cm, to contain, for example, a total volume of 1 ml of fluid.

The delivery device 400 also includes an activation mechanism 488 in the form of button that can be used to activate the insertion mechanism and/or the actuator 402. The first portion 482, the second portion 484 and the top portion 486 of the housing 480 can be coupled together in a similar manner as with various embodiments of a delivery system described in the '771 patent incorporated by reference above. The first portion 482, the second portion 484 and the top portion 486 can be coupled, for example, with an adhesive, a snap fit coupling or other known coupling method. The first portion 482 can be adhered to a patient's body with an adhesive layer disposed on a bottom surface of the first portion 482.

To use the delivery device 400, the delivery device 400 is placed at a desired injection site on a patient's body and adhesively attached thereto. With the fluid source 404 disposed within the housing 480 (e.g., inserted into the housing by the patient or predisposed), the patient can activate the insertion mechanism (not shown) to insert the fluid communicator (not shown) at the injection site. To activate the insertion mechanism to insert the fluid communicator (not shown) into a patient's body, the activation mechanism 488 (e.g., button) can be moved from an off position to an on position, which will cause the fluid communicator to penetrate the patient's skin at the treatment site.

The electrochemical actuator 402 can be activated after the insertion mechanism has been activated and the fluid communicator is inserted into the patient's body. Alternatively, in some embodiments, the electrochemical actuator 402 can be activated simultaneously with activation of the insertion mechanism. For example, when the insertion mechanism is activated it can be configured to activate a trigger mechanism (not shown) that communicates with the electrochemical actuator 402. For example, such a trigger mechanism can complete the electric circuit (as described above) and cause the electrochemical actuator 402 to start discharging. As the electrochemical actuator 402 discharges, the actuator 402 will displace and exert a force on the transfer structure 416, which in turn will exert a force on the fluid source 404, thereby compressing the fluid source 404 between the transfer structure 416 and the second portion 484 of the housing 480 and causing a volume of fluid within the fluid source 404 to be expelled into the patient.

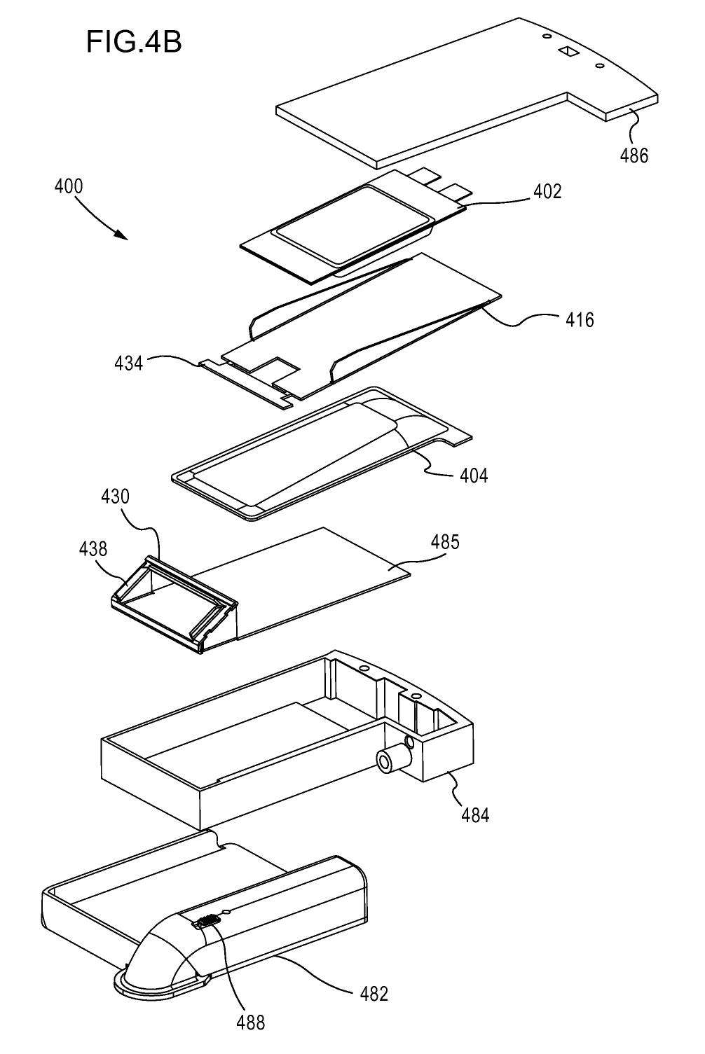

FIGS. 5A and 5B are schematic illustrations showing the difference between a mode of operation of an unclamped electrochemical actuator 502, shown in FIG. 5A, and a mode of operation of the electrochemical actuator 502 with one end clamped within a clamping mechanism 530, shown in FIG. 5(b). In this example, if in each scenario the actuator 502 has the same overall applied load (i.e., F1=F2), the electrochemical actuator 502 will undergo the same angular deflection θ₁=θ₂ in both the clamped and unclamped configurations. The resulting vertical displacement Δh, however, will be greater for the clamped actuator, Δh₂ than for the unclamped actuator, Δh₁.

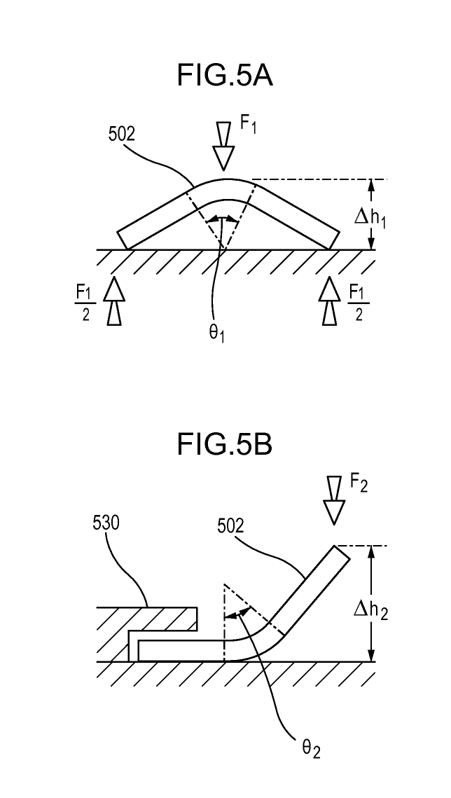

FIGS. 6A and 6B are schematic illustrations showing the difference between a mode of operation of an unclamped electrochemical actuator 602, shown in FIG. 6A, and a mode of operation of the electrochemical actuator 602 with one end clamped within a clamping mechanism 630, shown in FIG. 6B. This example illustrates a scenario where the actuator when clamped can achieve the same vertical deflection as when unclamped (i.e., Δh₂−Δh₁), but with a corresponding smaller angular deflection θ₁>θ₂. Thus, in this example, a greater overall vertical displacement rate can be achieved. As with the previous example, the actuator has the same overall applied load (i.e., F2=2(F1/2)=F1) in both the clamped and unclamped configurations.

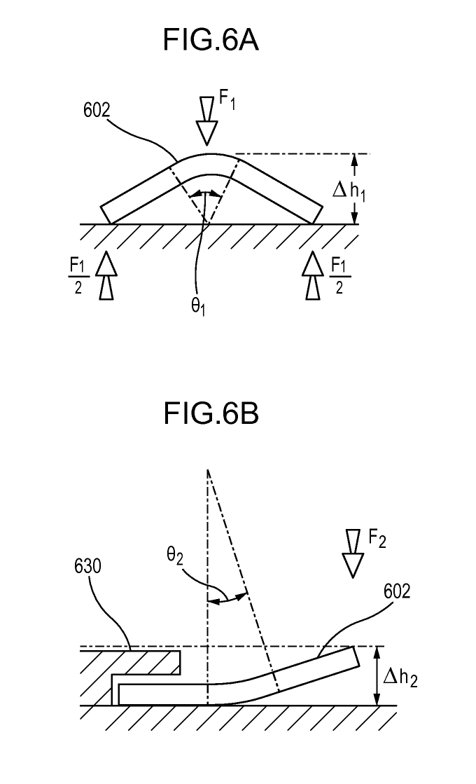

FIGS. 7A and 7B are schematic illustrations showing an embodiment of an electrochemical actuator 702 including optional external legs 726 and 728. FIG. 7A illustrates the electrochemical actuator 702 in a ready position prior to activation, and FIG. 7B illustrates the actuator 702 during activation. The external legs 726 and 728 are each coupled to an opposite end of the actuator 702 and external leg 726 is also coupled to a clamping mechanism 730. As shown, electronics 732 can be integrated into one of the legs (e.g., leg 726) that can include some or all of the necessary drive circuitry, communication units, as well as a switch to activate motion as needed. When the actuator 702 is activated, the actuator 702 will bend or deflect as shown in FIG. 7B.

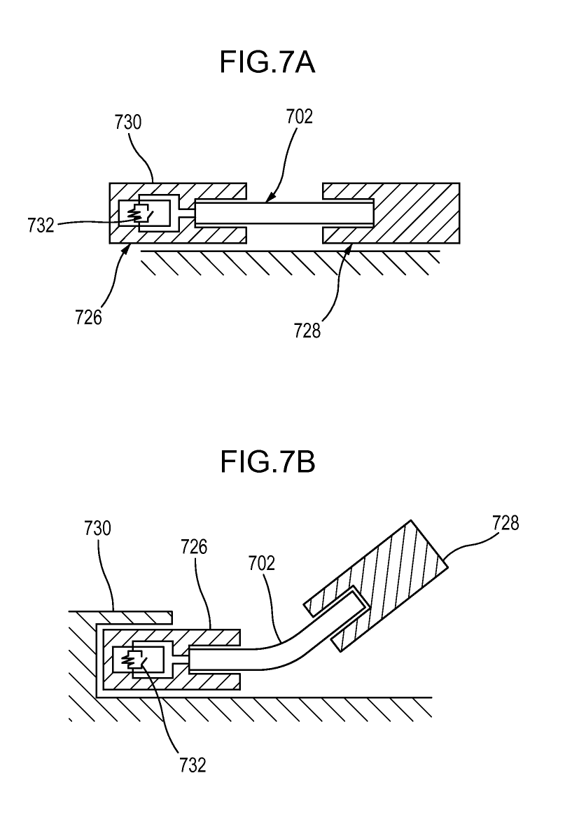

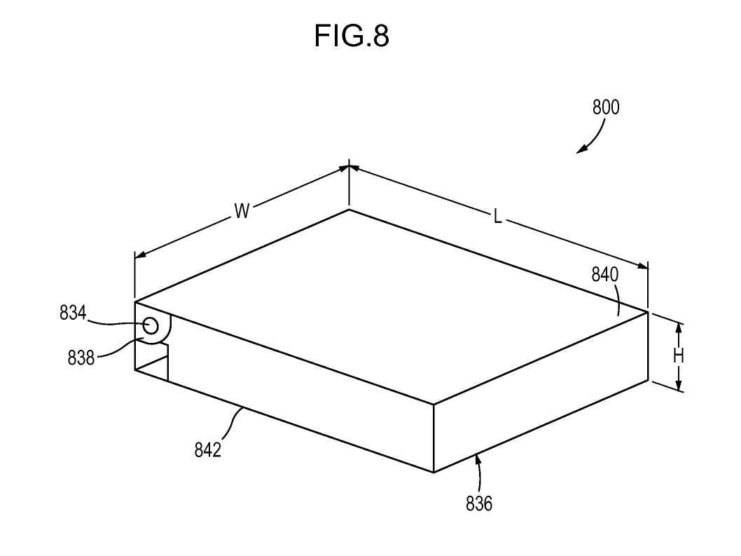

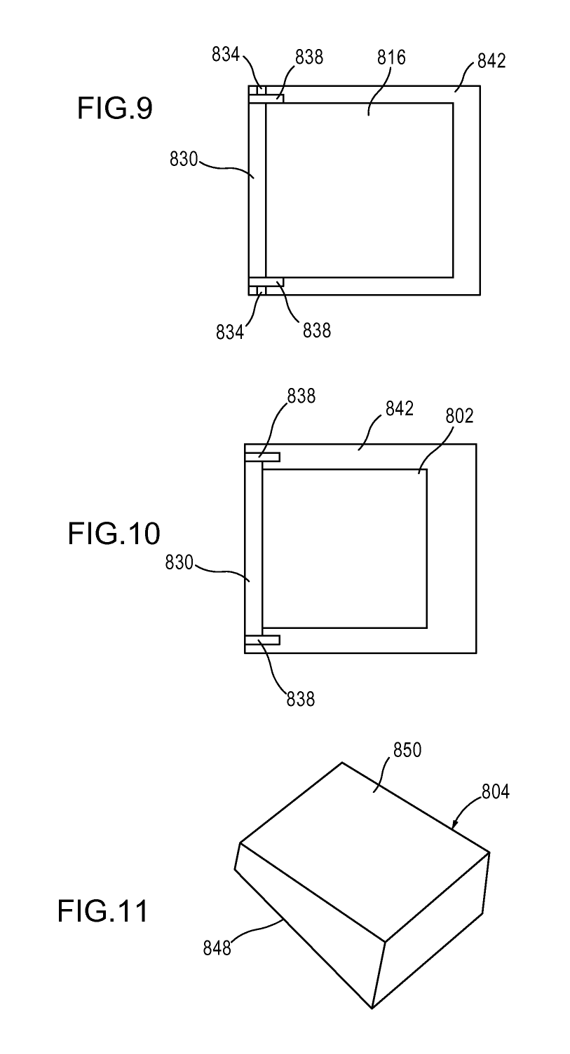

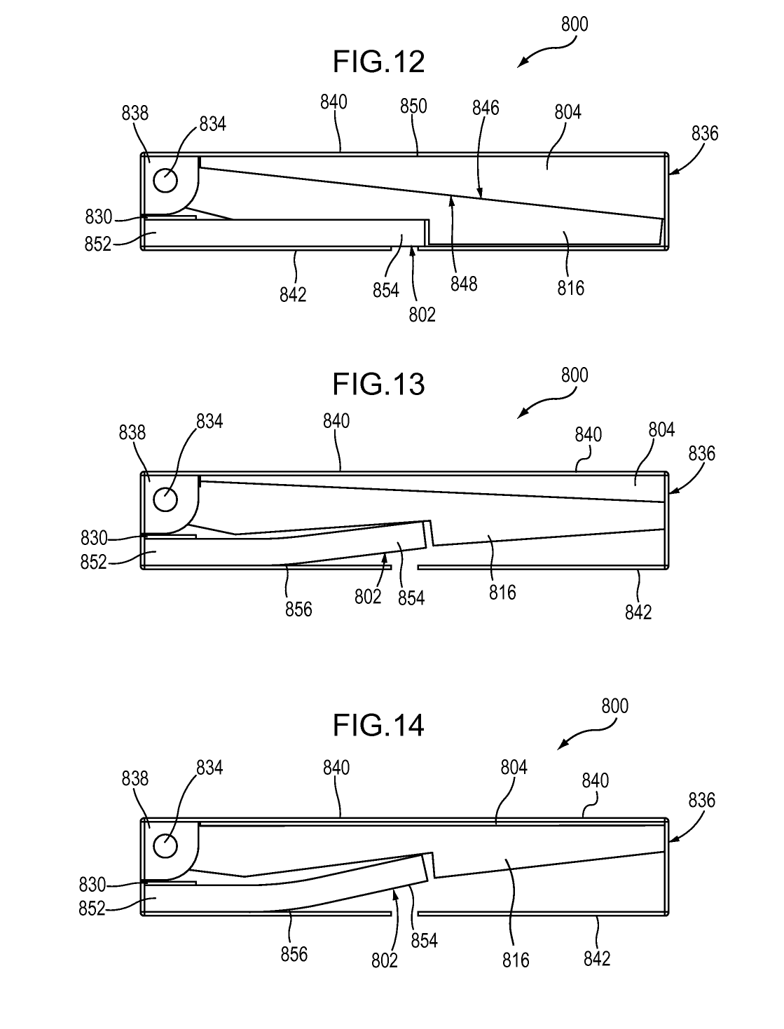

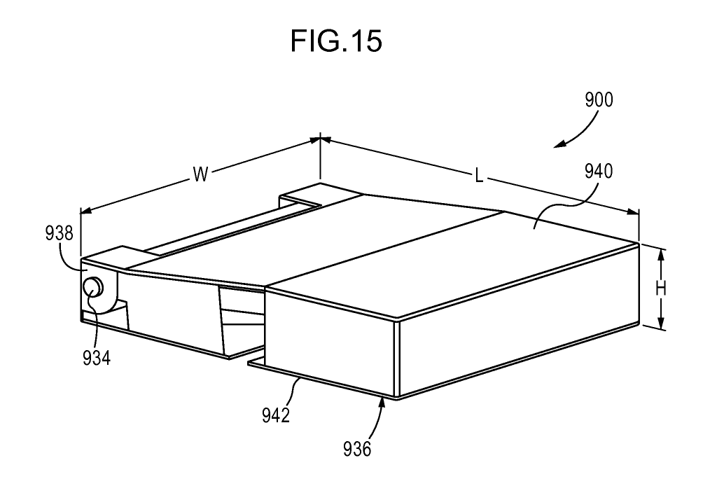

FIGS. 8-14 are schematic illustrations of a portion of an embodiment of a delivery system 800. In this embodiment, the delivery device 800 includes an electrochemical actuator 802, a transfer structure 816, and a fluid source 804. The transfer structure 816 includes pivot pins 834 that are pivotally coupled to a housing 836 at pivot mounting supports 838 (see e.g., FIG. 9) and is cantilevered at least partially over the electrochemical actuator 802. The fluid source 804 is disposed above the transfer structure 816, as shown in FIGS. 12-14. The transfer structure 816 includes a top surface 846 configured to contact a bottom surface 848 of the fluid source 804 (see, e.g., FIG. 11). In this embodiment, the bottom surface 848 of the fluid source 804 is angled relative to a top surface 850 of the fluid source 804, as shown in the side views of FIGS. 12-14. The housing 836 includes an upper wall portion 840 that is parallel to a bottom wall portion 842. In this embodiment, the top surface 850 of the fluid source 804 is parallel with the upper wall portion 840 and parallel to the lower wall portion 842 of the housing 836. The lower wall portion 842 can be adhered to a patient's body with an adhesive layer disposed on a bottom surface of the bottom wall portion 842.

The upper wall portion 840 and the lower wall portion 842 of the housing 836 can be coupled together in a similar manner as with various embodiments of a delivery system described in the '771 patent incorporated by reference above. For example, the upper wall portion 840 can be snapped or locked onto the bottom wall portion 842. In some embodiments, the upper wall portion 840 and the bottom wall portion 842 can be adhesively coupled together. The upper wall portion 840 and the bottom wall portion 842 collectively define an interior region of the housing 836 in which various components of the delivery device 800 are disposed. The housing 836 can have, for example, a length L of 1.93 inches, a width W of 1.53 inches and a depth or height H of 0.36 inches as shown in FIG. 8.

The electrochemical actuator 802 is constrained (e.g., clamped, attached, fixed) within a clamping mechanism 830 at a first end or edge portion 852 and free at an opposite second end or edge portion 854 such that when the electrochemical actuator 802 is activated, the actuator 802 will displace or bend in a pivotal manner about a pivot location 856, as shown in FIGS. 13 and 14. As discussed above, by constraining the actuator 802 on one end, its useful displacement can be increased and in some cases nearly doubled. The location of the point of contact where the actuator 802 pushes on the transfer structure 816 can leverage the energy (force and displacement) of the actuator 802 to determine in part, the force and motion capability of the transfer structure 816 that can be transferred to the fluid source 804.

In use, when the electrochemical actuator 802 is actuated (e.g., discharges), the free end portion 854 of the electrochemical actuator 802 will displace and exert a force on the transfer structure 816, as shown in FIG. 13. The transfer structure 816 will in turn exert a force on the fluid source 804 to pump a volume of fluid out of the fluid source 804. When the electrochemical actuator 802 has completed discharging and/or the transfer structure 816 and/or electrochemical actuator 802 have reached a limit on which they can be displaced, as shown in FIG. 14, the fluid source 804 will be substantially empty (e.g., fluid has been expelled out of the fluid source). In one example, the stroke or duration of actuation of the electrochemical actuator 802 can be, for example, 1.27 mm over for example, a period of 3.4 hours.

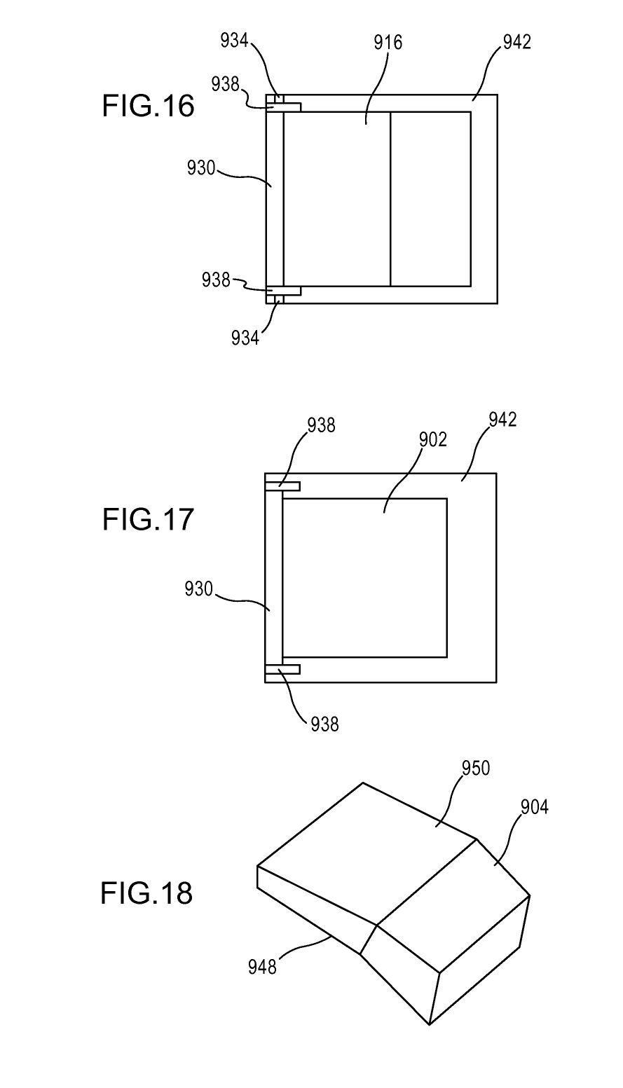

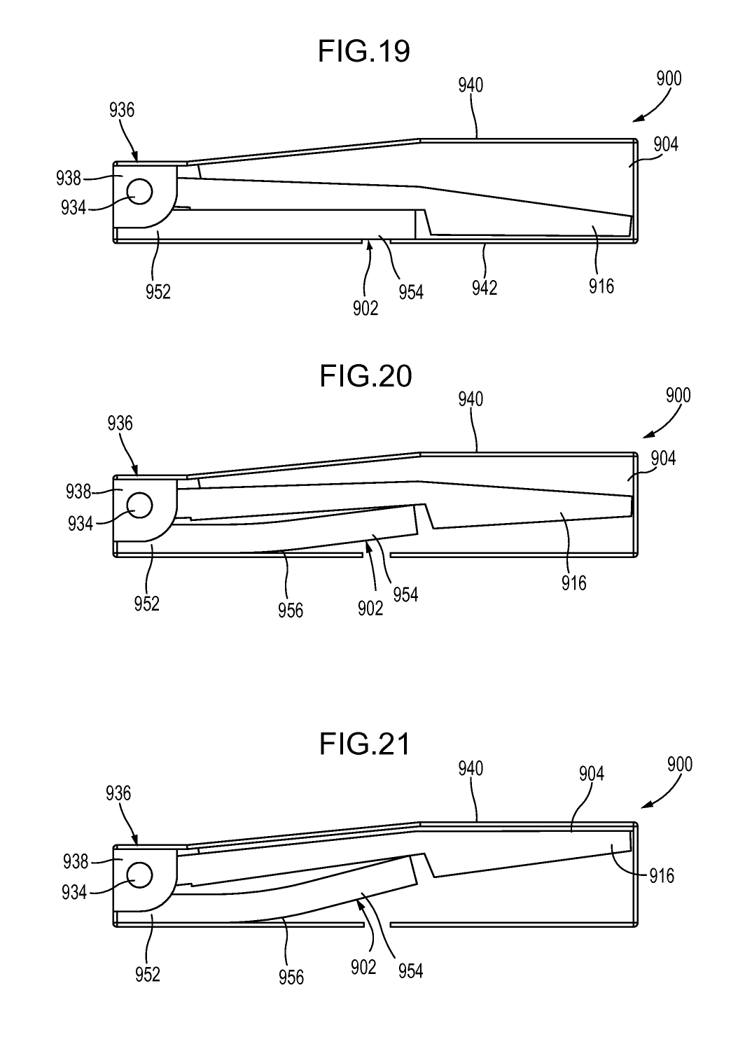

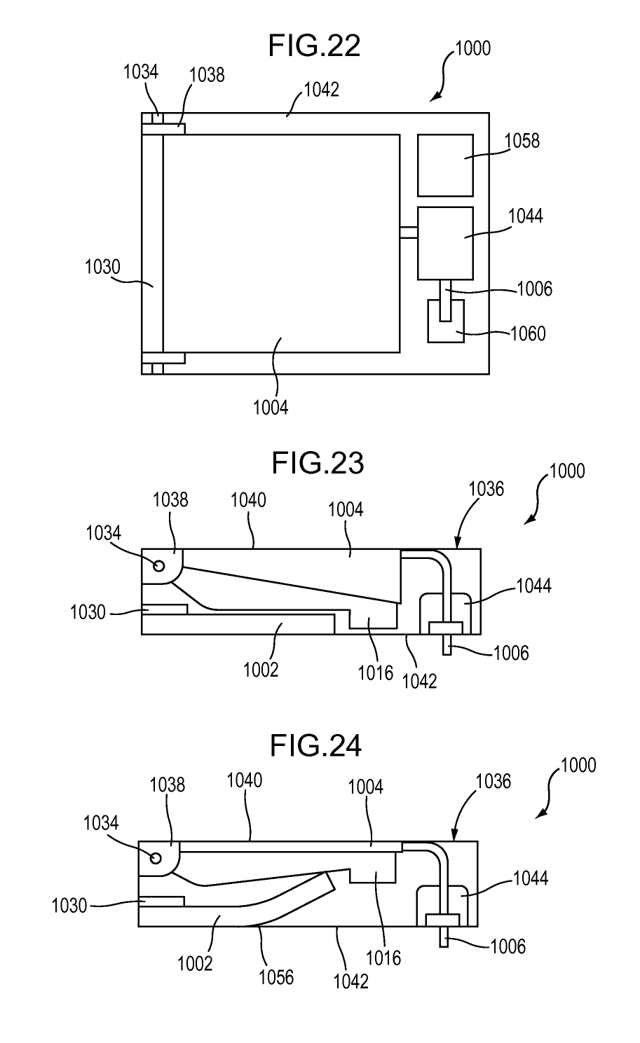

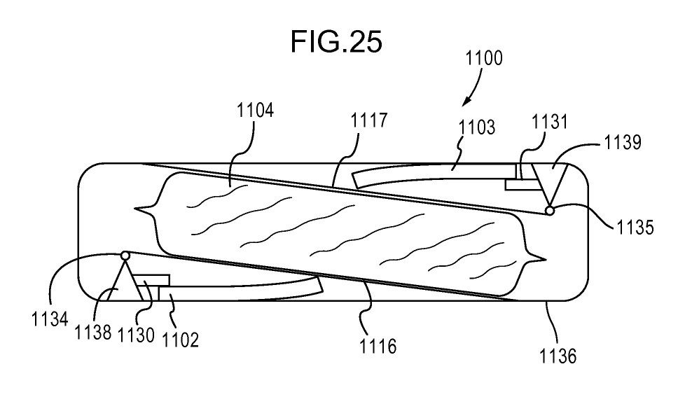

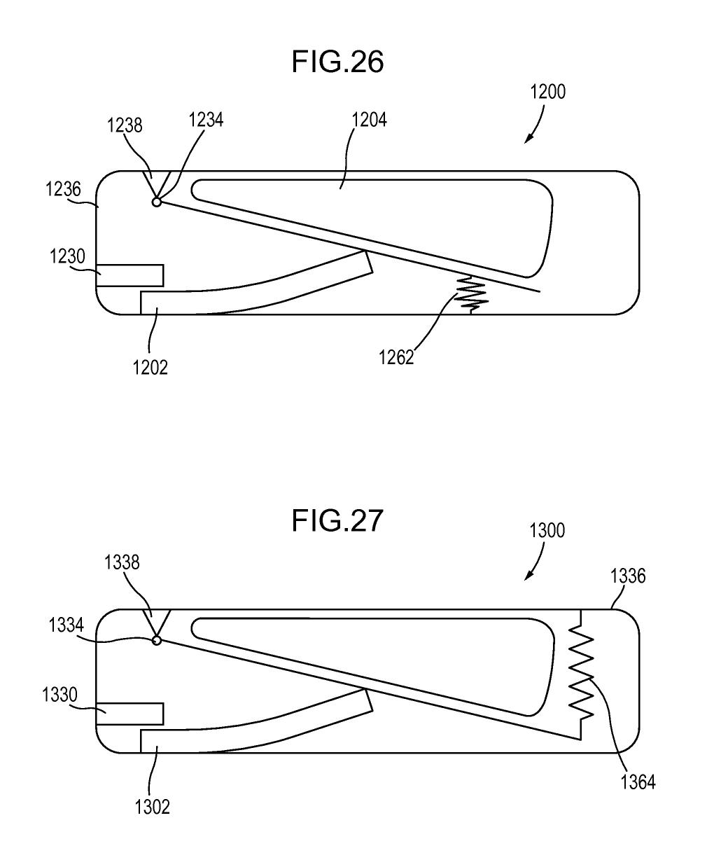

FIGS. 15-21 are schematic illustrations of a portion of another embodiment of a delivery system 900. As with the previous embodiment, the delivery system 900 includes an electrochemical actuator 902, a transfer structure 916 and a fluid source 904. The transfer structure 916 is pivotally mounted to a housing 936 at pivot mounting supports 938, and the transfer structure 916 is cantilevered at least partially over the electrochemical actuator 902. The fluid source 904 is disposed above the transfer structure 916. In this embodiment, the transfer structure 916 has an angled or bent top surface 946 configured to contact a corresponding angled bottom surface 948 of the fluid source 904, as shown in FIGS. 19-21. The fluid source 904 also includes an angled top surface 950 that corresponds to an angled upper wall portion 940 of the housing 936.

The upper wall portion 940 and the lower wall portion 942 of the housing 936 can be coupled together as described above for previous embodiments to collectively define an interior region of the housing 936. The lower wall portion 942 can be adhered to a patient's body with an adhesive layer disposed on a bottom surface of the bottom wall portion 942. The housing 936 can have, for example, a length L of 1.8 inches, a width W of 1.53 inches and a depth or height H of 0.36 inches at its widest point as shown in FIG. 15.

As with the previous embodiment, the electrochemical actuator 902 is constrained (e.g., clamped, attached, fixed) within a clamping mechanism 930 at a first end or edge portion 952 and includes a free end at an opposite second end or edge portion 954 such that when the electrochemical actuator 902 is activated, the actuator 902 will displace or bend in a pivotal manner about a pivot location 956, as shown in FIGS. 20 and 21. In use, when the electrochemical actuator 902 is actuated (e.g., discharges), the free end of the electrochemical actuator 902 will displace and exert a force on the transfer structure 816 as shown in FIG. 20. The transfer structure 916 will in turn exert a force on the fluid source 904 to pump a volume of fluid out of the fluid source 904.

FIG. 21 illustrates the delivery device 900 when the electrochemical actuator 902 has completed discharging and/or the transfer structure 916 and/or electrochemical actuator 902 have reached a limit on which they can be displaced. As shown in FIG. 21, when the electrochemical actuator 902 has completed its actuation, the fluid source 904 has been emptied (e.g., fluid has been expelled out of the fluid source). The stroke or duration of actuation of the electrochemical actuator 902 can be, for example, 1.5 mm over for example, a 4 hour period. The stroke and duration of activation described for delivery device 900 and delivery device 800 described above are just example actuation parameters, as other embodiments can have different actuation parameters depending on the particular configuration and desired output.

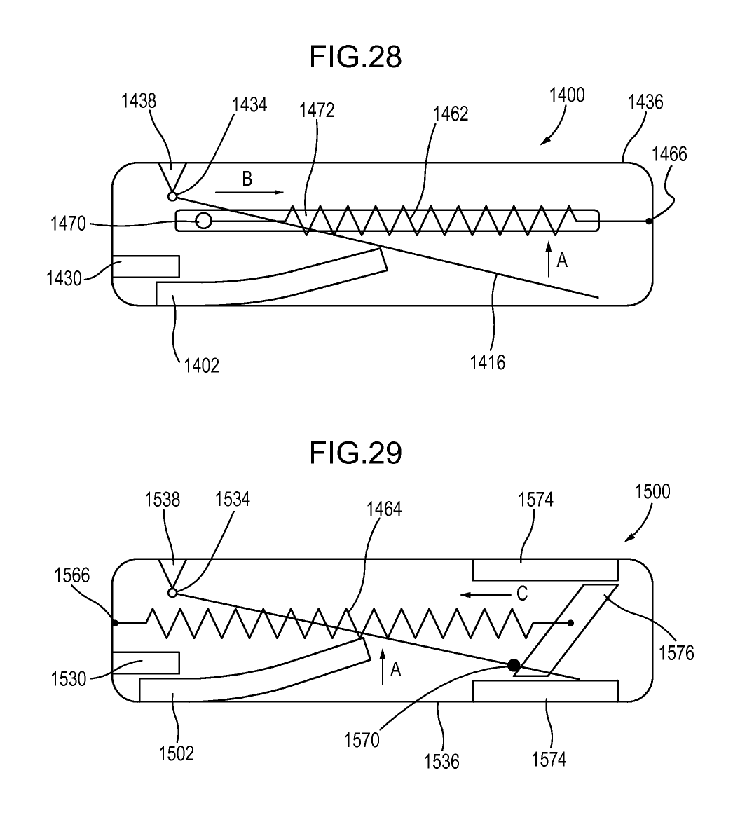

FIGS. 22-24 illustrate an embodiment of a drug delivery device that includes a clamped actuator as described herein. A delivery device 1000 includes a housing 1036, a fluid source 1004, a fluid communicator 1006, an electrochemical actuator 1002, a transfer structure 1016, a fluid communicator insertion mechanism 1044, and associated electronics 1058. The housing includes an upper wall portion 1040 and a lower wall portion 1042 that can be coupled together as described for previous embodiments. The lower wall portion 1042 can be adhered to a patient's body with an adhesive layer disposed on a bottom surface of the bottom wall portion 1042. FIG. 22 is a top view of the delivery device 1000 with the top wall portion 1042 removed for illustration purposes.

In this embodiment, the fluid communicator 1006 is in the form of a cannula that can be inserted into a patient's body using the insertion mechanism 1044. For example, the insertion mechanism 1044 can insert the fluid communicator 1006 through an opening 1060 defined in the lower wall portion 1042 of the housing 1036. In alternative embodiments, a separate insertion mechanism can be used. The fluid communicator 1006 can be placed in fluid communication with the fluid reservoir 1004 such that it can communicate the fluid within the fluid reservoir 1004 to the patient. For example, the insertion mechanism 1044 can be configured to puncture the fluid reservoir 1004 upon activation to create a fluid path between the fluid reservoir 1004 and the fluid communicator 1006.

In use, the delivery device 1000 can be attached to a patient's body and the insertion mechanism 1044 can be activated. Activation of the insertion mechanism 1044 can be achieved by actuating an activation mechanism (not shown). The activation mechanism can be a switch, button, pull-tab, etc. The insertion mechanism 1044 can also be used to trigger activation of the electrochemical actuator 1002 upon insertion of the fluid communicator 1006. In some embodiments, a secondary activation mechanism (not shown) is provided.

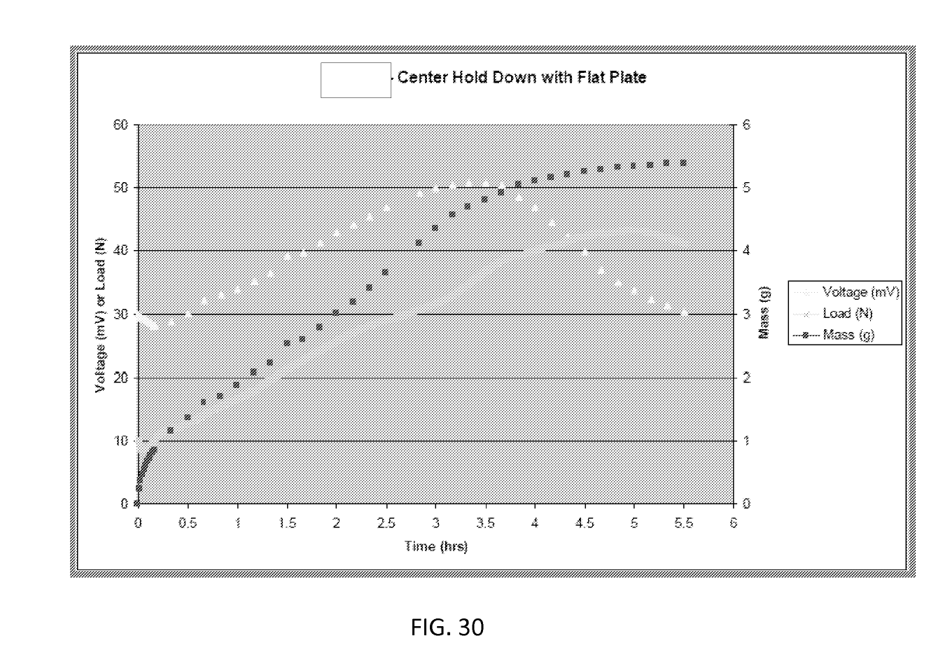

FIG. 23 illustrates the delivery device 1000 when the electrochemical actuator 1002 is in a charged state, and the fluid communicator 1006 has been inserted into the patient's body. In this configuration, the delivery device is in a ready mode. As described above, the electrochemical actuator can be triggered to begin discharging upon insertion of the fluid communicator 1006 or with a secondary mechanism. In either case, as described previously, when the electrochemical actuator 1002 is activated (e.g. the electrochemical actuator 1002 is discharging), the actuator 1002 will be displaced or bend about a pivot location 1056 as shown in FIG. 24. As the actuator 1002 is rotated upward, it contacts the transfer structure 1016 and causes it to pivot upward. This action in turn applies a force to the fluid reservoir 1004, squeezing the fluid reservoir 1004 between the transfer structure 1016 and the upper wall portion 1040 of the housing 1036. The fluid in the fluid reservoir 1004 will be pumped or expelled out of the fluid reservoir 1004, through the fluid communicator 1006 and into the patient's body.

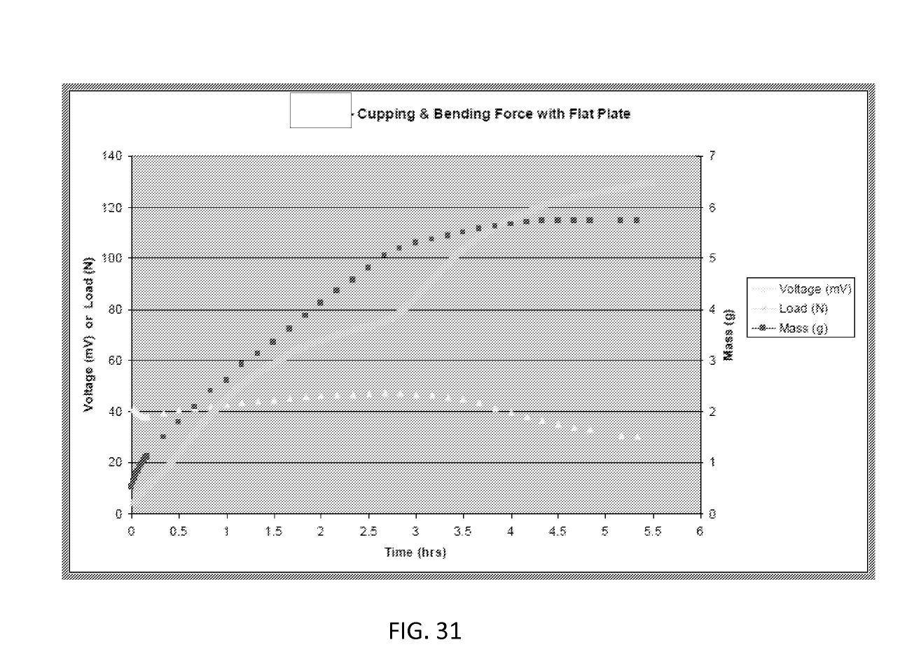

FIG. 25 is a schematic illustration of a delivery device 1100 according to another embodiment. The delivery device 1100 includes a double-cantilevered actuator arrangement that includes a first actuator 1102 and a second actuator 1103 that are each clamped on one end with a clamp mechanism 1130 and 1131, respectively. The first and second actuators 1102 and 1103 can each be, for example, an electrochemical actuator as described for other embodiments. A first transfer structure 1116 is pivotally coupled to a housing 1136 via a first pivot mounting support 1138 at a pivot location 1134, and a second transfer structure 1117 is pivotally coupled to the housing 1136 via a second pivot mounting support 1139 at a pivot location 1135. A fluid source 1104 is disposed between the first transfer structure 1116 and the second transfer structure 1117.

In this embodiment, as the first actuator and the second actuator 1103 are actuated (e.g., discharged), the first actuator 1102 can push on first transfer structure 1116 and the second actuator 1103 can push on the second transfer structure 1117, such that a volume of fluid within the fluid reservoir 1104 is emptied during the pivoting motion or the first and second transfer structures 1116 and 1117. The double actuators can provide for a greater force to be applied to the fluid reservoir 1104 and/or greater displacement. This configuration of a double cantilevered actuator arrangement may be desirable, for example, to enable larger drug volume payloads that have certain requirements on dispense rate. In some embodiments, doubling the reservoir thickness and using such an arrangement can enable delivering a doubled drug amount in the same amount of time. This configuration can also lend itself to other optimizations available, for example, by changing actuator size, clamp location, push location and reservoir size. It can also benefit from increased voltage available (as shown in FIG. 25(b)) from series connection of battery/actuator leads during discharge.

FIGS. 26-29 are schematic illustrations of embodiments of a delivery device that includes an electrochemical actuator used in conjunction with a mechanical spring(s). The mechanical spring can aid the dispense action in the early phases of function, thereby reducing loads on the electrochemical actuator in its early stages of discharge. This can also result in positive effects on its internal function, such as, for example, easier development of the lower density structure that causes deflection of the actuator.

In some embodiments, coupling springs with an electrochemical actuator can also help ensure that the pumping of the delivery device is initiated immediately upon actuation of the system (i.e., to supplement pumping during any stroke initiation delay that the electrochemical actuator may have). Another advantage of coupling a spring with an electrochemical actuator is to achieve more complicated delivery profiles, such as, for example, a bolus plus baseline delivery. By controlling when the power of the spring is unleashed, the bolus may be provided at any time during the delivery (e.g., beginning, middle, or end). In some embodiments, there may also be an advantage to releasing the spring toward the end of delivery to aid in complete emptying of the reservoir. In some embodiments, a delivery device can include a spring that acts alone to dispense the fluid (without the use of an actuator).

As shown in FIG. 26, a delivery device 1200 includes an actuator 1202 (e.g., an electrochemical actuator), clamped on one end with a clamping mechanism 1230. The clamping mechanism 1230 is coupled to a housing 1236 and a transfer structure 1216 is pivotally coupled to the housing 1236 with a mounting structure 1238 at a pivot location 1234. A fluid reservoir 1204 is disposed above the transfer structure 1216, and a compression spring 1262 is coupled to the housing 1236 and to the transfer structure 1216, as shown in FIG. 26. In this embodiment, the spring 1262 is positioned within the delivery device 1200 such that it can push on the transfer structure 1216 once compressed. Placement of the spring 1262 at different locations along a length of the transfer structure 1216 can change the applied torque of the spring 1262. Springs of different compressed size can be used depending on the initial location of the spring 1262. Springs placed closer to the pivot location 1234 can produce lower torque and vice-versa if placed toward the moving end (e.g., the free end). The spring 1262 can be, for example, an option wave, or a Belleville spring which can provide for more optimal use of space within the delivery device 1200.

In alternative embodiments, an extension spring can be used that is placed on the upper portion of the delivery device as shown in FIG. 27. A delivery device 1300 can be similarly configured as delivery device 1200 and includes an actuator 1302, a fluid reservoir 1304, a clamping mechanism 1330 coupled to a housing 1336, and a transfer structure 1316 pivotally coupled to the housing 1336 via a mounting structure 1338 at pivot location 1334. In this embodiment, an extension spring 1264 is coupled to the housing 1336 and to the transfer structure 1316 such that the spring 1364 can pull on the transfer structure 1316 with the same effect on force balance.

In another alternative embodiment, a delivery device can include a spring used in conjunction with an electrochemical actuator that is positioned along a length of the delivery device (as opposed to along its thickness as shown in FIGS. 26 ad 27). As shown in FIG. 28, a delivery device 1400 includes an actuator 1402, a fluid reservoir (not shown), a clamping mechanism 1430 coupled to a housing 1436, and a transfer structure 1416 pivotally coupled to the housing 1436 via a mounting structure 1438 at pivot location 1434. The delivery device 1400 also includes an extension spring 1464 movably coupled to the housing 1436. A first end of the spring 1464 is coupled to a side wall of the housing 1436 at 1466, and a second end of the spring 1464 includes a low-friction roller member 1470 that is movably disposed within a channel 1472 defined in a back wall of the housing 1436.

In use, when the actuator 1402 is actuated, the actuator 1402 pushes the transfer structure 1416 upward as described for previous embodiments. Substantially simultaneously, the spring can pull the roller member 1470 in a direction of arrow B causing the free end of the transfer structure 1416 to move upward in a direction of arrow A. Specifically, the spring 1464 can pull the roller member 1470 within the channel 1472 such that the roller member 1470 moves in a direction of arrow B and contacts and interfaces with a bottom surface of the transfer structure 1416 along a side edge of the transfer structure 1416, and pulls the transfer structure 1416 upward. The effectiveness of the spring 1464 can be higher upon initial activation of delivery device as the force angles are higher, and a greater portion of the spring force is directed perpendicular to the bottom surface of the transfer structure 1416. In alternative embodiments, the same result can be accomplished with a compression spring placed on the other side of the transfer structure 1416.

In another embodiment, a delivery device can use a drive wedge to push on a roller member that is attached to the longitudinal side edges of a transfer structure as shown in FIG. 29. In this embodiment, a delivery device 1500 includes an actuator 1502, a fluid reservoir (not shown), a clamping mechanism 1530 coupled to a housing 1536, a transfer structure 1516 pivotally coupled to the housing 1536 via a mounting structure 1538 at pivot location 1534, and an extension spring 1564. A first end of the spring 1564 is coupled to a wall of the housing 1536 at 1566, and a second end of the spring 1564 is coupled to a drive wedge 1576. The drive wedge 1576 is movably disposed between two guides 1574 and is constrained to ride/move between slide surfaces of the guides 1574 to minimize friction and restrict motion to a direction that is parallel to a longitudinal axis of the spring 1564.

A roller member 1570 is coupled to the drive wedge 1576 such that it can be moved along the longitudinal side edges of the transfer structure 1516 as the delivery device is actuated. Specifically, the drive wedge 1576 can be angled such that a vertical force is imparted by the roller member 1570 on a bottom surface of the transfer structure 1516 along a side edge of the transfer structure as the spring 1564 pulls the drive wedge 1576 in a direction of arrow C toward the pivot location 1534. This action will cause the free end of the transfer structure 1516 to move upward in a direction of arrow A. In some embodiments, the same result can be accomplished with a compression spring coupled on the other side of the drive wedge 1576. In some embodiments, the drive wedge 1576 can have a non-linear shape to change applied forces at portions of the stroke of the delivery device.

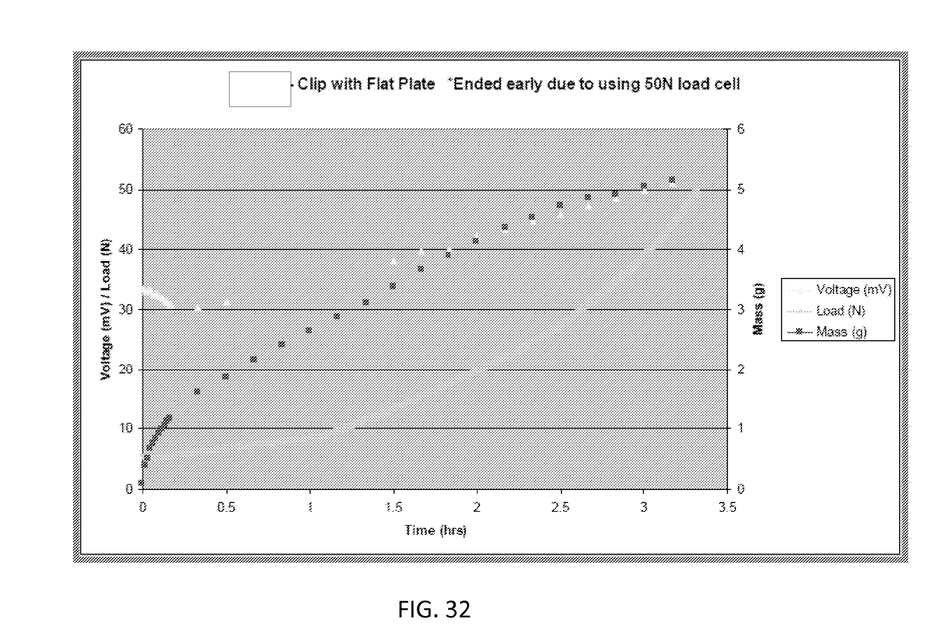

FIGS. 30-32 each illustrate the results of testing of an electrochemical actuator clamped or held at different locations on the electrochemical actuator. In the test, a rectangular actuator approximately 22 mm in width and 26 mm in length was clamped at one end, while the free end engaged a transfer structure which in turn engaged a fluid reservoir. The reservoir contained approximately 6 gm of fluid. As the actuator actuated, it curled longitudinally, so that the free end was displaced in a direction to apply force to the reservoir through the transfer structure and expel fluid from the reservoir. The displacement or stroke of the actuator corresponded to the amount of fluid expelled from the reservoir, i.e. in different tests the amount of fluid expelled at any given point corresponded to an amount of displacement of the free end of the actuator. Approximately 4 mm of the clamped end of the actuator was held in the clamp, and was fixed by an Instron machine, which measured the amount of force required to maintain the clamped end of the actuator in position. The graphs in FIGS. 30-32 shown the force applied to the fixed end of the actuator (“Load,” measured in Newtons), the cumulative amount of fluid expelled from the reservoir (“Mass,” measured in grams), and the voltage of the electrochemical actuator (“Voltage,” measured in milliVolts), all as a function of time (measured in hours).

FIG. 30 illustrates the results of a test where the actuator was held or clamped only near the center of the fixed end of the electrochemical actuator. In this example, the maximum hold-down force applied to the actuator is approximately 45-50 N. This force essentially represents the force required to oppose the resistance of the reservoir to displacement of the free end of the actuator. As shown in FIG. 30, the actuation pump-out is relatively slow, i.e. about 5 hours were required for the free end of the actuator to displace the transfer structure sufficiently to expel approximately 5.5 gm of fluid from the reservoir. This is attributed in part to the corners of the actuator being able to curl up on either side.

FIG. 31 illustrates the results of a test where the actuator is held or clamped all along the edge of the fixed end of the electrochemical actuator, to help prevent or limit curling of the actuator. In this example, the maximum hold-down force is substantially higher (approximately 130 N) than in the preceding test, which reflects the additional force required to resist the lateral curling of the corners of the fixed end. However, the actuator pump-out rate is about 3.5 hours to cover the same target distance (i.e. to expel approximately 5.5 gm of fluid) as the preceding test. FIG. 31 shows that the additional force needed to constrain bending the short axis (e.g., the width) of the actuator is approximately 80 N (about 130 N in this test as compared to about 50 N in the preceding test).

FIG. 32 illustrates the results of a test where the actuator was constrained along the edge of the fixed end of the electrochemical actuator with a c-shaped clip to prevent or limit lateral curling of the actuator. In this example, the maximum hold-down force is essentially the same as in the first test (approximately 50 N), because the lateral curling of the fixed end of the actuator is resisted by the clip, rather than by the Instron machine. The actuator pump-out rate, however, is about the same as the second test, i.e., approximately 5.2 gm of fluid was expelled in approximately 3.5 hours.

In some embodiments, a second clip can be used along the edge of the unconstrained end of the actuator, opposite the constrained end, to inhibit curling of the “free” corners of the actuator. In some cases, this can force or cause all of the “bending” of the actuator to occur at the center line and can result in faster bending.

A delivery device as described herein may be used to deliver a variety of drugs according to one or more release profiles. For example, the drug may be delivered according to a relatively uniform flow rate, a varied flow rate, a preprogrammed flow rate, a modulated flow rate, in response to conditions sensed by the device, in response to a request or other input from a user or other external source, or combinations thereof. Thus, embodiments of the delivery device may be used to deliver drugs having a short half-life, drugs having a narrow therapeutic window, drugs delivered via on-demand dosing, normally-injected compounds for which other delivery modes such as continuous delivery are desired, drugs requiring titration and precise control, and drugs whose therapeutic effectiveness is improved through modulation delivery or delivery at a non-uniform flow rate. These drugs may already have appropriate existing injectable formulations.

For example, the delivery devices may be useful in a wide variety of therapies. Representative examples include, but are not limited to, opioid narcotics such as fentanyl, remifentanyl, sufentanil, morphine, hydromorphone, oxycodone and salts thereof or other opioids or non-opioids for post-operative pain or for chronic and breakthrough pain; NonSteroidal Antinflamatories (NSAIDs) such as diclofenac, naproxen, ibuprofin, and celecoxib; local anesthetics such as lidocaine, tetracaine, and bupivicaine; dopamine antagonists such as apomorphine, rotigotine, and ropinerole; drugs used for the treatment and/or prevention of allergies such as antihistamines, antileukotrienes, anticholinergics, and immunotherapeutic agents; antispastics such as tizanidine and baclofin; insulin delivery for Type 1 or Type 2 diabetes; leutenizing hormone releasing hormone (LHRH) or follicle stimulating hormone (FSH) for infertility; plasma-derived or recombinant immune globulin or its constituents for the treatment of immunodeficiency (including primary immunodeficiency), autoimmune disorders, neurological and neurodegenerative disorders (including Alzheimer's Disease), and inflammatory diseases; apomorphine or other dopamine agonists for Parkinson's disease; interferon A for chronic hepatitis B, chronic hepatitis C, solid or hematologic malignancies; antibodies for the treatment of cancer; octreotide for acromegaly; ketamine for pain, refractory depression, or neuropathic pain; heparin for post-surgical blood thinning; corticosteroid (e.g., prednisone, hydrocortisone, dexamethasone) for treatment of MS; vitamins such as niacin; Selegiline; and rasagiline. Essentially any peptide, protein, biologic, or oligonucleotide, among others, that is normally delivered by subcutaneous, intramuscular, or intravenous injection or other parenteral routes, may be delivered using embodiments of the devices described herein. In some embodiments, the delivery device can be used to administer a drug combination of two or more different drugs using a single or multiple delivery port and being able to deliver the agents at a fixed ratio or by means enabling the delivery of each agent to be independently modulated. For example, two or more drugs can be administered simultaneously or serially, or a combination (e.g. overlapping) thereof.

In some embodiments, the delivery device may be used to administer ketamine for the treatment of refractory depression or other mood disorders. In some embodiments, ketamine may include either the racemate, single enantiomer (R/S), or the metabolite (wherein S-norketamine may be active). In some embodiments, the delivery devices described herein may be used for administration of Interferon A for the treatment of hepatitis C. In one embodiment, a several hour infusion patch is worn during the day or overnight three times per week, or a continuous delivery system is worn 24 hours per day. Such a delivery device may advantageously replace bolus injection with a slow infusion, reducing side effects and allowing the patient to tolerate higher doses. In other Interferon A therapies, the delivery device may also be used in the treatment of malignant melanoma, renal cell carcinoma, hairy cell leukemia, chronic hepatitis B, condylomata acuminata, follicular (non-Hodgkin's lymphoma, and AIDS-related Kaposi's sarcoma.

In some embodiments, a delivery device as described herein may be used for administration of apomorphine or other dopamine agonists in the treatment of Parkinson's Disease (“PD”). Currently, a bolus subcutaneous injection of apomorphine may be used to quickly jolt a PD patient out of an “off” state. However, apomorphine has a relatively short half-life and relatively severe side effects, limiting its use. The delivery devices described herein may provide continuous delivery and may dramatically reduce side effects associated with both apomorphine and dopamine fluctuation. In some embodiments, a delivery device as described herein can provide continuous delivery of apomorphine or other dopamine agonist, with, optionally, an adjustable baseline and/or a bolus button for treating an “off” state in the patient. Advantageously, this method of treatment may provide improved dopaminergic levels in the body, such as fewer dyskinetic events, fewer “off” states, less total time in “off” states, less cycling between “on” and “off” states, and reduced need for levodopa; quick recovery from “off” state if it occurs; and reduced or eliminated nausea/vomiting side effect of apomorphine, resulting from slow steady infusion rather than bolus dosing.

In some embodiments, a delivery device as described herein may be used for administration of an analgesic, such as morphine, hydromorphone, fentanyl or other opioids, in the treatment of pain. Advantageously, the delivery device may provide improved comfort in a less cumbersome and/or less invasive technique, such as for post-operative pain management. Particularly, the delivery device may be configured for patient-controlled analgesia.

### CONCLUSION

While various embodiments of the invention have been described above, it should be understood that they have been presented by way of example only, and not limitation. Where methods and steps described above indicate certain events occurring in certain order, those of ordinary skill in the art having the benefit of this disclosure would recognize that the ordering of certain steps may be modified and that such modifications are in accordance with the variations of the invention. Additionally, certain of the steps may be performed concurrently in a parallel process when possible, as well as performed sequentially as described above. The embodiments have been particularly shown and described, but it will be understood that various changes in form and details may be made.

For example, although various embodiments have been described as having particular features and/or combinations of components, other embodiments are possible having any combination or sub-combination of any features and/or components from any of the embodiments described herein. For example, although some embodiments were not described as including an insertion mechanism, an activation mechanism, electrical circuitry, etc., it should be understood that those embodiments of a delivery device can include any of the features, components and/or functions descried herein for other embodiments. In addition, the specific configurations of the various components can also be varied. For example, the size and specific shape of the various components can be different than the embodiments shown, while still providing the functions as described herein.

What is claimed is:

1. An apparatus, comprising:
a reservoir configured to contain a fluid;
a fluid communicator configured to be placed in fluid communication with the reservoir; and
an actuator having a first end, a second end, and a medial portion between the first end and the second end, the actuator being configured so that when actuated, the actuator bends in the medial portion and produces a displacement of the second end in a first direction relative to the first end,
the actuator being disposed with the first end constrained, the second end unconstrained, and oriented so that when the actuator is actuated, the second end is displaced toward the fluid reservoir and exerts a force on the reservoir such that fluid within the reservoir is communicated through the fluid communicator.

2. The apparatus of claim 1, further comprising:
a transfer structure disposed between the actuator and the reservoir, the transfer structure configured to contact the reservoir upon actuation of the actuator.

3. The apparatus of claim 1, wherein the actuator is an electrochemical actuator.

4. The apparatus of claim 1, further comprising:
a housing configured to be removably coupled to a patient, the housing defining an interior region, the actuator and reservoir disposed within the housing.

5. The apparatus of claim 1, further comprising:
a housing configured to be removably coupled to a patient; and
an insertion mechanism coupled to the housing, the insertion mechanism configured to insert the fluid communicator into the patient.

6. The apparatus of claim 1, further comprising:
a transfer structure disposed between the actuator and the reservoir, the transfer structure having a top surface configured to contact the reservoir upon actuation of the actuator, the top surface of the transfer structure being non-parallel to a top surface of the actuator.

7. The apparatus of claim 1, wherein the reservoir is wedge shaped prior to actuation of the actuator.

8. An apparatus, comprising:
a housing removably couplable to a user;
a reservoir configured to contain a fluid and disposed within the housing;
an actuator having a constrained first end portion and an unconstrained second end portion; and
a transfer structure disposed between the actuator and the reservoir, the transfer structure having a first end portion pivotally coupled to the housing and an unconstrained second end portion, the transfer structure having a surface configured to contact the reservoir upon actuation of the actuator,
the actuator being configured such that when actuated, a force is exerted by the second end portion of the actuator onto the transfer structure and the unconstrained second end portion of the transfer structure pivots about a pivot location and exerts a force on the reservoir such that fluid within the reservoir is communicated out of the reservoir.

9. The apparatus of claim 8, wherein the actuator is an electrochemical actuator.

10. The apparatus of claim 8, further comprising:
a fluid communicator configured to be placed in fluid communication with the reservoir such that when the actuator is actuated, fluid in the reservoir is communicated into the user via the fluid communicator.

11. The apparatus of claim 8, wherein the actuator is a first actuator, the force exerted by the actuator is a first force, the apparatus further comprising:
a second actuator having a constrained first end portion and an unconstrained second end portion, the second actuator configured to exert a second force, different than the first force, on the reservoir.

12. The apparatus of claim 11, wherein the transfer structure is a first transfer structure, the apparatus further comprising:
a second transfer structure disposed between the second actuator and the reservoir, the second actuator configured to exert the second force on the second transfer structure in an opposite direction as the first force.

13. The apparatus of claim 11, wherein the transfer structure is a first transfer structure, the apparatus further comprising:
a second transfer structure disposed between the second actuator and the reservoir, the second transfer structure having a first end portion pivotally coupled to the housing and an unconstrained second end portion, the second transfer structure having a surface configured to contact the reservoir upon actuation of the actuator,
the second actuator being configured such that when actuated, the second force is exerted by the second end portion of the actuator onto the transfer structure and the unconstrained second end portion of the transfer structure pivots about a pivot location and exerts a force on the reservoir such that fluid within the reservoir is communicated out of the reservoir.

14. The apparatus of claim 8, wherein the force exerted by the actuator is a first force, the apparatus further comprising:
a spring coupled to the transfer structure, the spring configured to exert a second force onto the transfer structure such that the unconstrained second end portion of the transfer structure is moved toward the reservoir.

15. The apparatus of claim 8, wherein the force exerted by the actuator is a first force, the apparatus further comprising:
a spring coupled to the transfer structure, a first end portion of the spring being slidably disposed within a channel defined by the housing, the spring configured to exert a second force onto the transfer structure such that the second end portion of the transfer structure is moved toward the reservoir.

16. An apparatus, comprising:
a housing removably couplable to a user;
a reservoir configured to contain a fluid and disposed within the housing;
an actuator having a constrained first end portion and an unconstrained second end portion;
a transfer structure disposed between the actuator and the reservoir,
the actuator being configured so that when actuated, a first force is exerted by the actuator onto the transfer structure; and
a spring coupled to the transfer structure, the spring configured to exert a second force onto the transfer structure, the first force and the second force collectively configured to cause the transfer structure to exert a force on the reservoir such that fluid within the reservoir is communicated out of the reservoir.

17. The apparatus of claim 16, wherein the actuator is an electrochemical actuator.

18. The apparatus of claim 16, wherein a first end portion of the spring is coupled to a roller member configured to slidably move within a channel defined by the housing, the roller member configured to exert the second force onto the transfer structure.

19. The apparatus of claim 16, wherein a first end portion of the spring is coupled to a drive wedge configured to slidably move relative to the transfer structure such that a roller member coupled to the drive wedge exerts the second force on the transfer structure.

20. The apparatus of claim 16, wherein the spring is a compression spring.

21. The apparatus of claim 16, wherein the spring is an extension spring.

22. The apparatus of claim 16, wherein the actuator has a medial portion between the first end portion and the second end portion, the actuator being configured so that when actuated, the medial portion of the actuator bends and imparts the first force on the transfer structure.
